(19) United States
(12) Patent Application Publication
(10) Pub. No.: US 2020/0087597 A1
(43) Pub. Date: Mar. 19, 2020

(54) Encapsulated Solid Enzyme Product

(71) Applicant: Novozymes A/S, Bagsvaerd (DK)

(72) Inventors: Jia Ju, Beijing (CN); Zhenwei Li, Beijing (CN); Wei Wei, Beijing (CN); Ole Simonsen, Soeborg (DK); Kim Bruno Andersen, Vaerloese (DK); Wenjie Du, Beijing (CN)

(73) Assignee: Novozymes A/S, Bagsvaerd (DK)

(21) Appl. No.: 16/468,614

(22) PCT Filed: Dec. 21, 2017
(86) PCT No.: PCT/CN2017/117706

§ 371 Date: Jun. 11, 2019

(30) Foreign Application Priority Data
Dec. 28, 2016 (CN) PCT/CN2016/112581

(51) Int. Cl. C11D 3/386; C12M 1/00; C12N 9/96; C12N 1/20; C11D 17/04; C11D 3/37; C11D 3/38

(52) U.S. Cl. CPC C11D 3/38609; C12M 23/14; C12N 9/96; C12N 1/20; C12N 2500/50; C11D 3/3753; C11D 3/3707; C11D 3/381; C11D 17/044

(57) ABSTRACT

A pouch comprising a wall forming a chamber, the wall comprising a water-soluble polymer, and the chamber containing a particulate solid comprising an enzyme, and a liquid comprising a surfactant and/or a polyol. A method of preparing a pouch and a method of preparing an enzyme-containing detergent composition are disclosed.

### REFERENCE TO A SEQUENCE LISTING

This application contains a Sequence Listing in computer readable form, which is incorporated herein by reference.

### FIELD OF THE INVENTION

The present invention relates to pouches containing an enzyme or microorganism, methods of producing the pouches, and methods of preparing detergent compositions using the pouches.

### BACKGROUND

Detergent compositions often comprise enzymes alongside other components such as surfactants. A variety of different enzymes may be used in such detergent compositions in order to help clean substrates, such as laundry. The enzymes may include proteases to break down proteinaceous material, lipases to break down fatty materials, and amylases to break down carbohydrate-based material. Similarly, WO 2012/112718 describes the use of microorganisms to control malodor in cleaning machines and cleaning processes. These biological active detergent ingredients are often added in liquid form as a solution or suspension during the preparation of the detergent composition, which may also contain water and surfactants, amongst other components.

### SUMMARY OF THE INVENTION

The present invention seeks to provide an improved process for the preparation of enzyme or microorganism containing detergent compositions.

According to the present invention, there is provided a pouch comprising a wall forming a chamber, the wall comprising a water-soluble polymer, and the chamber containing a particulate solid comprising an enzyme or microorganism, and a liquid comprising a surfactant and/or a polyol.

In an embodiment, the weight of the pouch is more than 50 g (such as 50 g-25 kg), preferably more than 100 g, more than 250 g, or more than 500 g. Advantageously, the pouch comprises more than 0.5% w/w active enzyme protein, preferably more than 1% w/w active enzyme protein, or more than 5% w/w active enzyme protein.

Preferably, the enzyme is selected from a protease (e.g., subtilisin or metalloprotease), lipase, cutinase, amylase, carbohydrase, cellulase, pectinase, mannanase, arabinase, galactanase, xanthanase, xylanase, DNAse, perhydrolase, oxidoreductase (e.g., laccase, peroxidase, peroxygenase and/or haloperoxidase); preferably a protease (e.g., subtilisin or metalloprotease), lipase, amylase, cellulase, pectinase, and/or mannanase.

Conveniently, the particulate solid comprises a spray dried enzyme.

Preferably, the pouch comprises more than one enzyme. Conveniently, the pouch comprises more than one chamber.

Preferably, the microorganism is a bacterium, fungus or yeast, such as a dehydrated bacterium or yeast. In an embodiment, the microorganism is a microbial spore, such as a bacterial (endo)spore. In a preferred embodiment, the microorganism is a Bacillus endospore, such as an endospore of Bacillus subtilis, Bacillus licheniformis, Bacillus amyloliquefaciens, or Bacillus megaterium.

Advantageously, the surfactant comprises a non-ionic surfactant, preferably a polyoxyethylene alkyl ether surfactant. Preferably, the polyoxyethylene alkyl ether surfactant is a Softanol™ surfactant, preferably Softanol™ 90.

Conveniently, the polyol comprises glycerol, (mono, di, or tri) propylene glycol, sugar alcohol (such as sorbitol), polypropylene glycol (preferably a polypropylene glycol having a molecular weight in the range of 200-800 (PPG200-800)), and/or polyethylene glycol (preferably a polyethylene glycol having a molecular weight in the range of 200-800 (PEG200-800)).

Conveniently, the degree of hydrolysis of the PVA is from about 75% to about 99%. Advantageously, the wall thickness is from about 20 to about 5000 μm, preferably from about 50 to about 3000 μm, more preferably from about 50 to about 2000 μm, more preferably from about 50 to about 1000 μm, more preferably from about 100 to about 2000 μm, and most preferably from about 100 to about 1000 μm.

Preferably, the chamber further contains one or more additive. Conveniently, the one or more additives comprises a surfactant, a polyol, and/or an enzyme inhibitor.

According to an aspect of the invention, there is provided a method of preparing a pouch, the method comprising forming a wall which forms a chamber, the wall comprising a water-soluble polymer; providing a particulate solid comprising an enzyme, and a liquid comprising a surfactant and/or a polyol in the chamber; and sealing the chamber to form the pouch. Preferably, the pouch is as defined above.

According to an aspect of the invention, there is provided a method of preparing an enzyme-containing detergent composition comprising adding a pouch to an aqueous composition, wherein the pouch comprises a wall forming a chamber, the wall comprising a water-soluble polymer, and the chamber containing a particulate solid comprising an enzyme, and a liquid comprising a surfactant and/or a polyol. Preferably, the pouch is added to an aqueous composition consisting of one or more detergent ingredients, such as surfactant(s) and/or builder(s). Conveniently, the pouch is as defined above. Advantageously, the aqueous composition is agitated to aid the dissolution of the pouch.

According to an aspect of the invention, there is provided a detergent composition obtainable by the method described above.

Other aspects and embodiments of the invention are apparent from the description and examples.

The invention will now be described, by way of example, with reference to the accompanying figures, in which:

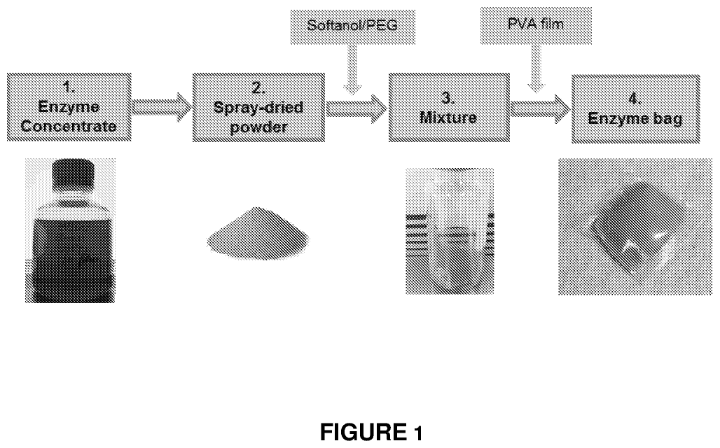

FIG. 1 is a schematic diagram showing the preparation of a pouch containing an enzyme-containing material; and

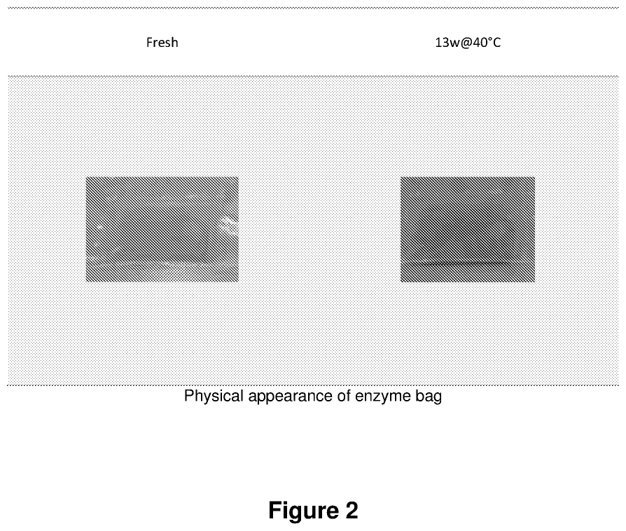

FIG. 2 shows the appearance of a pouch containing an enzyme-containing material before and after storage tests.

### DETAILED DESCRIPTION

The present invention is aimed at providing a product for use in the preparation of such compositions which results in an improved process. The invention relates to a pouch comprising the enzyme or microorganism, with the pouch being made of a material that allow it to be added to a detergent composition to release its contents. The pouch may comprise a wall forming a chamber, with the wall comprising a water soluble polymer. The chamber contains a particulate solid which comprises an enzyme, and a liquid comprising a surfactant and/or a polyol. In an aqueous environment, such as a detergent composition, the wall dissolves at least enough to release its contents, which can then result in the formation of an enzyme- or microorganism-containing detergent composition. The enzyme or microorganism is provided in a particulate solid form, and the pouch may additionally comprise additives to aid in the overall process.

The pouch contains the enzyme or microorganism in a particulate solid. This means that the solid is present as a powder or particles, rather than as a tablet or unitary solid form. The pouch also contains a liquid that contains a surfactant and/or a polyol. Some of the enzyme may be solubilized by the liquid, thus making the liquid a saturated solution with respect to the enzyme. Preferably, the liquid contains a surfactant and a polyol. The particulate solid and the liquid are mixed together in the chamber. The pouch can contain more than one enzyme. This can be achieved by the chamber containing particulate solid material that contains more than one enzyme (either in separate solid particles, or in the same solid particles). Also, the pouch can contain more than one chamber. In one embodiment, a first chamber can contain a first enzyme, and a second chamber can contain a second enzyme.

The additives may provide stability to the enzyme and other components, and may also aid in the dissolution of the enzyme into the detergent composition.

The invention has numerous benefits. The formulation of the enzyme pouch is relatively simple and has commercial advantages as compared to the preparation and use of the equivalent amount of enzyme in a liquid form.

Also, the invention allows the use of enzymes in the form of a spray-dried material. The spray-dried material is preferably wetted by one or more additive to improve the properties of the material. For example, the one or more additive may improve the stability and/or dissolution properties of the pouch and/or material. A wetted spray-dried solid product comprising an enzyme can have superior stability as compared to other enzyme-containing material, such as liquid formulations. This is particularly the case in terms of thermal stability, and so may be particularly useful in areas or countries which have relatively high temperatures. Also, the provision of enzymes in a pouch form allows the mixing or blending of different enzymes together. This allows accurate measurement and blending, and also allows the formation of a stable product. Also, there are benefits in terms of forming a blend of more than one enzyme in a convenient way that minimises interaction of the enzymes, and so minimises possible degradation, for example during storage.

For detergent producers, the enzyme pouch is easy to use and dose without the requirement for dosing or measurement equipment. So a pouch containing a particular amount of material can be added to a detergent composition in order to produce an enzyme-containing detergent composition.

The pouches can also contain a very high enzyme activity, and can do so at an overall production and distribution cost which is lower than the equivalent amount of enzyme provided in liquid form.

The pouches may be made of any material which provides stability to the contents, and also allows the pouch to release its contents, for example by dissolving, in the formation of a detergent composition.

The use of a pouch of the invention has safety advantages over the use of materials that can produce enzyme-containing dust. Such dust is potentially irritating or harmful if inhaled. The use of the pouches of the invention facilitates the easy and simple handling of enzymes without exposing people to this hazard.

Wall Forming a Chamber

The pouch according to the invention comprises a wall forming a chamber, the wall comprising a water-soluble polymer. Thus, the water-soluble polymer is a constituent of a water-soluble film, which is used to make the wall forming a chamber.

Water-soluble films, optional ingredients for use therein, and methods of making the same are well known in the art. In one class of embodiments, the water-soluble film includes PVA (polyvinylacetate). As used herein ‘PVA’ refers to polyvinlyacetate itself, and to a synthetic resin generally prepared by the alcoholysis, usually termed hydrolysis or saponification, of polyvinyl acetate. Fully hydrolyzed PVA, wherein virtually all the acetate groups have been converted to alcohol groups, (i.e. polyvinyl alcohol), is a strongly hydrogen-bonded, highly crystalline polymer which dissolves only in hot water—greater than about 60° C. If a sufficient number of acetate groups are allowed to remain after the hydrolysis of polyvinyl acetate, the PVA polymer is then known as partially hydrolyzed, it is more weakly hydrogen-bonded and less crystalline and is soluble in cold water—less than about 10° C. An intermediate cold/hot water-soluble film can include, for example, intermediate partially-hydrolyzed PVA (e.g., with degrees of hydrolysis of about 94% to about 98%), and is readily soluble only in warm water—e.g., rapid dissolution at temperatures of about 40° C. and greater. Both fully and partially hydrolyzed PVA types are commonly referred to as PVA homopolymers although the partially hydrolyzed type is technically a vinyl alcohol-vinyl acetate copolymer.

The degree of hydrolysis of the PVA included in the water-soluble films of the present disclosure can be about 75% to about 99%. As the degree of hydrolysis is reduced, a film made from the resin will have reduced mechanical strength but faster solubility at temperatures below about 20° C. As the degree of hydrolysis increases, a film made from the resin will tend to be mechanically stronger and the thermoformability will tend to decrease. The degree of hydrolysis of the PVA can be chosen such that the water-solubility of the resin is temperature dependent, and thus the solubility of a film made from the resin, compatibilizing agent, and additional ingredients is also influenced. In one class of embodiments the film is cold water-soluble. A cold water-soluble film, soluble in water at a temperature of less than 10° C., can include PVA with a degree of hydrolysis in a range of about 75% to about 90%, or in a range of about 80% to about 90%, or in a range of about 85% to about 90%. In another class of embodiments the film is hot water-soluble. A hot water-soluble film, soluble in water at a temperature of at least about 60° C., can include PVA with a degree of hydrolysis of at least about 98%.

Other film-forming resins for use in addition to or in an alternative to PVA can include, but are not limited to, modified polyvinyl alcohols, polyacrylates, water-soluble acrylate copolymers, polyacrylates, polyacryamides, polyvinyl pyrrolidone, pullulan, water-soluble natural polymers including, but not limited to, guar gum, xanthan gum, carrageenan, and starch, water-soluble polymer derivatives including, but not limited to, ethoxylated starch and hydroxypropylated starch, poly(sodium acrylamido-2-methylpropane sulfonate), polymonomethylmaleate, copolymers thereof, and combinations of any of the foregoing. In one class of embodiments, the film-forming resin is a terpolymer consisting of vinyl alcohol, vinyl acetate, and sodium acrylamido-2-methyl propanesulfonate.

The water-soluble resin can be included in the water-soluble film in any suitable amount, for example an amount in a range of about 35% w/w to about 90% w/w.

Water-soluble resins for use in the films described herein (including, but not limited to PVA resins) can be characterized by any suitable viscosity for the desired film properties, optionally a viscosity in a range of about 5.0 to about 30.0 cP, or about 10.0 cP to about 25 cP. The viscosity of a PVA resin is determined by measuring a freshly made solution using a Brookfield LV type viscometer with UL adapter as described in British Standard EN ISO 15023-2:2006 Annex E Brookfield Test method. It is international practice to state the viscosity of 4% aqueous polyvinyl alcohol solutions at 20° C. All PVA viscosities specified herein in cP should be understood to refer to the viscosity of 4% aqueous polyvinyl alcohol solution at 20° C., unless specified otherwise.

It is well known in the art that the viscosity of a PVA resin is correlated with the weight average molecular weight of the same PVA resin, and often the viscosity is used as a proxy for the weight average molecular weight. Thus, the weight average molecular weight of the water-soluble resin optionally can be in a range of about 35,000 to about 190,000, or about 80,000 to about 160,000. The molecular weight of the resin need only be sufficient to enable it to be molded by suitable techniques to form a thin plastic film.

The water-soluble films according to the present disclosure may include other optional additive ingredients including, but not limited to, plasticizers, surfactants, defoamers, film formers, antiblocking agents, internal release agents, anti-yellowing agents and other functional ingredients, for example in amounts suitable for their intended purpose.

Water is recognized as a very efficient plasticizer for PVA and other polymers; however, the volatility of water makes its utility limited since polymer films need to have at least some resistance (robustness) to a variety of ambient conditions including low and high relative humidity. Glycerol is much less volatile than water and has been well established as an effective plasticizer for PVA and other polymers.

Plasticizers for use in water-soluble films of the present disclosure include, but are not limited to, sorbitol, glycerol, diglycerol, propylene glycol, ethylene glycol, diethyleneglycol, triethylene glycol, tetraethyleneglycol, polyethylene glycols up to MW 400, 2 methyl 1, 3 propane diol, lactic acid, monoacetin, triacetin, triethyl citrate, 1,3-butanediol, trimethylolpropane (TMP), polyether triol, and combinations thereof. Polyols, as described above, are generally useful as plasticizers. As less plasticizer is used, the film can become more brittle, whereas as more plasticizer is used the film can lose tensile strength. Plasticizers can be included in the water-soluble films in an amount in a range of about 25 phr to about 50 phr, or from about 30 phr to about 45 phr, or from about 32 phr to about 42 phr, for example.

Surfactants for use in water-soluble films are well known in the art. Optionally, surfactants are included to aid in the dispersion of the resin solution upon casting. Suitable surfactants for water-soluble films of the present disclosure include, but are not limited to, dialkyl sulfosuccinates, lactylated fatty acid esters of glycerol and propylene glycol, lactylic esters of fatty acids, sodium alkyl sulfates, polysorbate 20, polysorbate 60, polysorbate 65, polysorbate 80, alkyl polyethylene glycol ethers, lecithin, acetylated fatty acid esters of glycerol and propylene glycol, sodium lauryl sulfate, acetylated esters of fatty acids, myristyl dimethylamine oxide, trimethyl tallow alkyl ammonium chloride, quaternary ammonium compounds, salts thereof and combinations of any of the forgoing. Thus, surfactants can be included in the water-soluble films in an amount of less than about 2 phr, for example less than about 1 phr, or less than about 0.5 phr, for example.

One type of secondary component contemplated for use is a defoamer. Defoamers can aid in coalescing of foam bubbles. Suitable defoamers for use in water-soluble films according to the present disclosure include, but are not limited to, hydrophobic silicas, for example silicon dioxide or fumed silica in fine particle sizes, including Foam Blast® defoamers available from Emerald Performance Materials, including Foam Blast® 327, Foam Blast® UVD, Foam Blast® 163, Foam Blast® 269, Foam Blast® 338, Foam Blast® 290, Foam Blast® 332, Foam Blast® 349, Foam Blast® 550 and Foam Blast® 339, which are proprietary, non-mineral oil defoamers. In embodiments, defoamers can be used in an amount of 0.5 phr, or less, for example, 0.05 phr, 0.04 phr, 0.03 phr, 0.02 phr, or 0.01 phr. Preferably, significant amounts of silicon dioxide will be avoided, in order to avoid stress whitening.

Processes for making water-soluble articles, including films, include casting, blow-molding, extrusion and blown extrusion, as known in the art. One contemplated class of embodiments is characterized by the water-soluble film described herein being formed by casting, for example, by admixing the ingredients described herein with water to create an aqueous mixture, for example a solution with optionally dispersed solids, applying the mixture to a surface, and drying off water to create a film. Similarly, other compositions can be formed by drying the mixture while it is confined in a desired shape.

In one contemplated class of embodiments, the water-soluble film is formed by casting a water-soluble mixture wherein the water-soluble mixture is prepared according to the steps of:

(a) providing a mixture of water-soluble resin, water, and any optional additives excluding plasticizers;
(b) boiling the mixture for 30 minutes;
(c) degassing the mixture in an oven at a temperature of at least 40° C.; optionally in a range of 40° C. to 90° C., e.g., about 65° C.;
(d) adding one or more plasticizers, and additional water to the mixture at a temperature of 65° C. or less; and
(e) stirring the mixture without vortex until the mixture appears substantially uniform in color and consistency; optionally for a time period in a range of 30 minutes to 90 minutes, optionally at least 1 hour; and
(f) casting the mixture promptly after the time period of stirring (e.g., within 4 hours, or 2 hours, or 1 hour).

The film is useful for creating a packet to contain an enzyme-containing composition, thereby forming a pouch. The film described herein can also be used to make a packet with two or more compartments made of the same film or in combination with films of other polymeric materials. Additional films can, for example, be obtained by casting, blow-molding, extrusion or blown extrusion of the same or a different polymeric material, as known in the art. In one type of embodiment, the polymers, copolymers or derivatives thereof suitable for use as the additional film are selected from polyvinyl alcohols, polyvinyl pyrrolidone, polyalkylene oxides, polyacrylic acid, cellulose, cellulose ethers, cellulose esters, cellulose amides, polyvinyl acetates, polycarboxylic acids and salts, polyaminoacids or peptides, polyamides, polyacrylamide, copolymers of maleic/acrylic acids, polysaccharides including starch and gelatin, natural gums such as xanthan, and carrageenans. For example, polymers can be selected from polyacrylates and water-soluble acrylate copolymers, methylcellulose, carboxymethylcellulose sodium, dextrin, ethylcellulose, hydroxyethyl cellulose, hydroxypropyl methylcellulose, maltodextrin, polymethacrylates, and combinations thereof, or selected from polyvinyl alcohols, polyvinyl alcohol copolymers and hydroxypropyl methyl cellulose (HPMC), and combinations thereof.

The pouches and/or packets of the present disclosure comprise at least one sealed compartment. Thus the pouches may comprise a single compartment or multiple compartments. The pouches may have regions with and without enzymes. In embodiments including multiple compartments, each compartment may contain identical and/or different compositions. In turn, in addition to the compositions of the invention that contain a solid comprising an enzyme, any such additional compositions may take any suitable form including, but not limited to liquid, solid and combinations thereof (e.g., a solid suspended in a liquid). In some embodiments, the pouches comprises a first, second and third compartment, each of which respectively contains a different first, second and third composition. In some embodiments, the compositions may be visually distinct as described in EP 2258820.

The compartments of multi-compartment pouches and/or packets may be of the same or different size(s) and/or volume(s). The compartments of the present multi-compartment pouches can be separate or conjoined in any suitable manner. In some embodiments, the second and/or third and/or subsequent compartments are superimposed on the first compartment. In one embodiment, the third compartment may be superimposed on the second compartment, which is in turn superimposed on the first compartment in a sandwich configuration. Alternatively the second and third compartments may be superimposed on the first compartment. However it is also equally envisaged that the first, second and optionally third and subsequent compartments may be attached to one another in a side by side relationship. The compartments may be packed in a string, each compartment being individually separable by a perforation line. Hence each compartment may be individually torn-off from the remainder of the string by the end-user.

The pouches and/or packets of the present disclosure may comprise one or more different films. For example, in single compartment embodiments, the packet may be made from one wall that is folded onto itself and sealed at the edges, or alternatively, two walls that are sealed together at the edges. In multiple compartment embodiments, the packet may be made from one or more films such that any given packet compartment may comprise walls made from a single film or multiple films having differing compositions. In one embodiment, a multi-compartment pouch comprises at least three walls: an outer upper wall; an outer lower wall; and a partitioning wall. The outer upper wall and the outer lower wall are generally opposing and form the exterior of the pouch. The partitioning wall is interior to the pouch and is secured to the generally opposing outer walls along a seal line. The partitioning wall separates the interior of the multi-compartment pouch into at least a first compartment and a second compartment.

Pouches and packets may be made using any suitable equipment and method. For example, single compartment pouches may be made using vertical form filling, horizontal form filling, or rotary drum filling techniques commonly known in the art. Such processes may be either continuous or intermittent. The film may be dampened, and/or heated to increase the malleability thereof. The method may also involve the use of a vacuum to draw the film into a suitable mold. The vacuum drawing the film into the mold can be applied for about 0.2 to about 5 seconds, or about 0.3 to about 3, or about 0.5 to about 1.5 seconds, once the film is on the horizontal portion of the surface. This vacuum can be such that it provides an under-pressure in a range of 10 mbar to 1000 mbar, or in a range of 100 mbar to 600 mbar, for example.

The molds, in which packets may be made, can have any shape, length, width and depth, depending on the required dimensions of the pouches. The molds may also vary in size and shape from one to another, if desirable. For example, the volume of the final pouches may be about 50 ml to about 25 l, or about 50 ml to 10 l, or about 100 ml to about 5000 ml, or about 100 ml to about 1000 ml, and that the mold sizes are adjusted accordingly.

In one embodiment, the packet includes a first and a second sealed compartment. The second compartment is in a generally superposed relationship with the first sealed compartment such that the second sealed compartment and the first sealed compartment share a partitioning wall interior to the pouch.

In one embodiment, the packet including a first and a second compartment further includes a third sealed compartment. The third sealed compartment is in a generally superposed relationship with the first sealed compartment such that the third sealed compartment and the first sealed compartment share a partitioning wall interior to the pouch.

In one embodiment, the single compartment or plurality of sealed compartments contains a composition. The plurality of compartments may each contain the same or a different composition.

Heat can be applied to the film in the process commonly known as thermoforming. The heat may be applied using any suitable means. For example, the film may be heated directly by passing it under a heating element or through hot air, prior to feeding it onto a surface or once on a surface. Alternatively, it may be heated indirectly, for example by heating the surface or applying a hot item onto the film. The film can be heated using an infrared light. The film may be heated to a temperature of at least 50° C., for example about 50° C. to about 150° C., about 50° C. to about 120° C., about 60° C. to about 130° C., about 90° C. to about 120° C., or about 60° C. to about 90° C.

Alternatively, the film can be wetted by any suitable means, for example directly by spraying a wetting agent (including water, a solution of the film composition, a plasticizer for the film composition, or any combination of the foregoing) onto the film, prior to feeding it onto the surface or once on the surface, or indirectly by wetting the surface or by applying a wet item onto the film.

Once a film has been heated and/or wetted, it may be drawn into an appropriate mold, preferably using a vacuum. The film can be thermoformed with a draw ratio of at least about 1.5, for example, and optionally up to a draw ratio of 2, for example. The filling of the molded film can be accomplished by utilizing any suitable means. In some embodiments, the most preferred method will depend on the product form and required speed of filling. In some embodiments, the molded film is filled by in-line filling techniques. The filled, open packets are then closed forming the pouches, using a second film, by any suitable method. This may be accomplished while in horizontal position and in continuous, constant motion. The closing may be accomplished by continuously feeding a second film, preferably water-soluble film, over and onto the open packets and then preferably sealing the first and second film together, typically in the area between the molds and thus between the packets.

Any suitable method of sealing the packet and/or the individual compartments thereof may be utilized. Non-limiting examples of such means include heat sealing, solvent welding, solvent or wet sealing, and combinations thereof. The water-soluble packet and/or the individual compartments thereof can be heat sealed at a temperature of at least 95° C., for example in a range of about 105° C. to about 145° C., or about 110° C. to about 140° C. Typically, only the area which is to form the seal is treated with heat or solvent. The heat or solvent can be applied by any method, typically on the closing material, and typically only on the areas which are to form the seal. If solvent or wet sealing or welding is used, it may be preferred that heat is also applied. Preferred wet or solvent sealing/welding methods include selectively applying solvent onto the area between the molds, or on the closing material, by for example, spraying or printing this onto these areas, and then applying pressure onto these areas, to form the seal. Sealing rolls and belts as described above (optionally also providing heat) can be used, for example.

The formed pouches may then be cut by a cutting device. Cutting can be accomplished using any known method. It may be preferred that the cutting is also done in continuous manner, and preferably with constant speed and preferably while in horizontal position. The cutting device can, for example, be a sharp item, or a hot item, or a laser, whereby in the latter cases, the hot item or laser ‘burns’ through the film/sealing area.

The different compartments of a multi-compartment pouches may be made together in a side-by-side style wherein the resulting, cojoined pouches may or may not be separated by cutting. Alternatively, the compartments can be made separately.

In some embodiments, pouches may be made according to a process including the steps of:

a) forming a first compartment (as described above);
b) forming a recess within some or all of the closed compartment formed in step (a), to generate a second molded compartment superposed above the first compartment;
c) filling and closing the second compartments by means of a third film;
d) sealing the first, second and third films; and
e) cutting the films to produce a multi-compartment pouch.

The recess formed in step (b) may be achieved by applying a vacuum to the compartment prepared in step (a).

In some embodiments, second, and/or third compartment(s) can be made in a separate step and then combined with the first compartment as described in EP 2088187 or WO 2009/152031.

In other embodiments, pouches may be made according to a process including the steps of:

a) forming a first compartment, optionally using heat and/or vacuum, using a first film on a first forming machine;
b) filling the first compartment with a first composition;
c) on a second forming machine, deforming a second film, optionally using heat and vacuum, to make a second and optionally third molded compartment;
d) filling the second and optionally third compartments;
e) sealing the second and optionally third compartment using a third film;
f) placing the sealed second and optionally third compartments onto the first compartment;
g) sealing the first, second and optionally third compartments; and
h) cutting the films to produce a multi-compartment pouch.

The first and second forming machines may be selected based on their suitability to perform the above process. In some embodiments, the first forming machine is preferably a horizontal forming machine, and the second forming machine is preferably a rotary drum forming machine, preferably located above the first forming machine.

It should be understood that by the use of appropriate feed stations, it may be possible to manufacture multi-compartment pouches incorporating a number of different or distinctive compositions and/or different or distinctive liquid, gel or paste compositions.

A preferred material for the formation of the walls of the pouch is PVA (polyvinylacetate) film. PVA film is used in the formation of various different products, including pouches. The grade, structure, and thickness of the film can be selected to provide the required dissolution characteristics. Some forms of PVA film dissolve more readily at lower temperature than other forms of film, which are intended for more rapid dissolution at higher temperatures. Also, the thickness of the films affects the speed of dissolution. Preferred types of PVA for use in the formation of pouches of the invention include material having a degree of hydrolysis of the PVA of from about 75% to about 99%.

The thickness of the film can vary depending upon the type of PVA film and the required dissolution characteristics. However, the thickness may be from about 20 to about 5000 μm, preferably from about 50 to about 3000 μm, more preferably from about 100 to about 2000 μm.

The pouch preferably contains one chamber to hold the enzyme material. However, it is contemplated that the pouch may contain more than one chamber, for example where two different enzyme materials are preferably kept apart during storage.

Enzyme(s)

The composition used in the pouch of the invention includes one or more enzymes, in particular enzymes suitable for including in laundry or dishwash detergents (detergent enzymes), such as a protease (e.g., subtilisin or metalloprotease), lipase, cutinase, amylase, carbohydrase, cellulase, pectinase, mannanase, arabinase, galactanase, xanthanase, xylanase, DNAse, perhydrolase, oxidoreductase (e.g., laccase, peroxidase, peroxygenase and/or haloperoxidase). Preferred detergent enzymes are protease (e.g., subtilisin or metalloprotease), lipase, amylase, lyase, cellulase, pectinase, mannanase, DNAse, perhydrolase, and oxidoreductases (e.g., laccase, peroxidase, peroxygenase and/or haloperoxidase); or combinations thereof. More preferred detergent enzymes are protease (e.g., subtilisin or metalloprotease), lipase, amylase, cellulase, pectinase, and mannanase; or combinations thereof.

The detergent enzyme(s) may be included in a detergent composition by adding separate additives containing one or more enzymes, or by adding a combined additive comprising all of these enzymes.

Proteases

The proteases for use in the present invention are serine proteases, such as subtilisins, metalloproteases and/or trypsin-like proteases. Preferably, the proteases are subtilisins or metalloproteases; more preferably, the proteases are subtilisins.

A serine protease is an enzyme which catalyzes the hydrolysis of peptide bonds, and in which there is an essential serine residue at the active site (White, Handler and Smith, 1973 “Principles of Biochemistry,” Fifth Edition, McGraw-Hill Book Company, NY, pp. 271-272). Subtilisins include, preferably consist of, the I-S1 and I-S2 sub-groups as defined by Siezen et al., Protein Engng. 4 (1991) 719-737; and Siezen et al., Protein Science 6 (1997) 501-523. Because of the highly conserved structure of the active site of serine proteases, the subtilisin according to the invention may be functionally equivalent to the proposed sub-group designated subtilase by Siezen et al. (supra).

The subtilisin may be of animal, vegetable or microbial origin, including chemically or genetically modified mutants (protein engineered variants), preferably an alkaline microbial subtilisin. Examples of subtilisins are those derived from Bacillus, e.g., subtilisin Novo, subtilisin Carlsberg, subtilisin BPN′, subtilisin 309, subtilisin 147 and subtilisin 168 (described in WO 89/06279) and Protease PD138 (WO 93/18140). Examples are described in WO 98/020115, WO 01/44452, WO 01/58275, WO 01/58276, WO 03/006602 and WO 04/099401.

Other examples of useful proteases are the variants described in WO92/19729, WO96/034946, WO98/20115, WO98/20116, WO99/011768, WO01/44452, WO03/006602, WO04/03186, WO04/041979, WO07/006305, WO11/036263, WO11/036264, especially the variants with substitutions in one or more of the following positions: 3, 4, 9, 15, 27, 36, 43, 57, 61, 62, 68, 76, 87, 95, 96, 97, 98, 99, 100, 101, 102, 103, 104, 106, 118, 120, 123, 128, 129, 130, 156, 158, 160, 161, 167, 170, 182, 185, 188, 191, 194, 195, 199, 204, 205, 206, 209, 212, 217, 218, 224, 232, 235, 236, 245, 248, 252, 261, 262, 274 and 275, using the BPN′ numbering. The protease may comprise a substitution at one or more positions corresponding to positions 171, 173, 175, 179, or 180 of SEQ ID NO: 3 of WO 2004/067737.

More preferred the protease variants may comprise one or more of the following substitutions: S3T, V4I, S9R, S9E, A15T, K27R, *36D, N43R, G61E, G61D, N62D, N62E, V68A, N76D, N87S,R, *97E, A98S, S99G,S99D, S99A, S99AD, S101E, S101D, S101G, S101M, S101N, S101R, S101H, S103A, V1041, V104Y, V104N, S106A, G118V, G118R, H120D, H120N, N123S, 5128L, P129Q, S130A, S156D, A158E, G160D, G160P, S161E, Y167A, R1705, Q182E, N185E, S188E, Q191N, A194P, G195E, V199M, N204D, V2051, Y209W, S212G, L217Q, L217D, N218D, N2185, A232V, K235L, Q236H, Q245R, N252K, N261W, N261D, N261E, L262E, L262D T274A, R275H (using BPN′ numbering).

Examples of commercially available proteases include those sold under the trade names Alcalase™ Relase™, Relase™ Ultra, Savinase™, Savinase™ Ultra, Duralase™, Durazym™ Everlase™, Primase™, Polarzyme™, Kannase™, Liquanase™, Liquanase™ Ultra, Ovozyme™, Coronase™, Coronase™ Ultra, Blaze™, Blaze Evity™ 100T, Blaze Evity™ 125T, Blaze Evity™ 150T, Neutrase™, Esperase™ Carnival™, Progress Uno™ and Progress Excel™ (Novozymes A/S); those sold under the tradename Maxatase™ Maxacal™, Puramax™, FN2™, FN3™, FN4™, Excellase™, Maxapem™ Purafect Ox™ Purafect OxP™, Effectenz™ P1050, Effectenz™ P1060, Excellenz™ P1000, Excellenz™ P1250, Eraser™, Preferenz™ P100, Purafect Prime™, Preferenz™ P110, Effectenz™ P1000, Purafect™, Effectenz™ P2000, Purafast™, Properase™, Opticlean™ and Optimase™ (Genencor/Danisco/DuPont); Axapem™ (Gist-Brocases N.V.); BLAP (sequence shown in FIG. 29 of U.S. Pat. No. 5,352,604) and variants hereof (Henkel AG); and KAP (Bacillus alkalophilus subtilisin) from Kao.

Lyases

The lyase may be a pectate lyase derived from Bacillus, particularly B. licherniformis or B. agaradhaerens, or a variant derived of any of these, e.g. as described in U.S. Pat. No. 6,124,127, WO 99/027083, WO 99/027084, WO 02/006442, WO 02/092741, WO 03/095638, Commercially available pectate lyases are XPect™; Pectawash™ and Pectaway™ (Novozymes A/S).

Mannanase

The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 99/064619. Commercially available mannanases are Mannaway™ (Novozymes A/S), and Mannastar™ (Dupont).

Cellulases

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. Nos. 4,435,307, 5,648,263, 5,691,178, 5,776,757 and WO 89/09259.

Especially suitable cellulases are the alkaline or neutral cellulases having color care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. Nos. 5,457,046, 5,686,593, 5,763,254, WO 95/24471, WO 98/12307 and PCT/DK98/00299.

Commercially available cellulases include Celluzyme™ Carezyme™ Carezyme Premium™ Whitezyme™ and Celluclean™ (Novozymes A/S); Clazinase™ Revitalenz™, and Puradax HA™ (DuPont); Biotouch™ DCL and FCL (AB Enzymes); and KAC-500(B)™ (Kao Corporation).

Lipases and Cutinases

Suitable lipases and cutinases include those of bacterial or fungal origin. Chemically modified or protein engineered mutants are included. Examples include lipase from Thermomyces, e.g., from T. lanuginosus (previously named Humicola lanuginosa) as described in EP 258 068 and EP 305 216, cutinase from Humicola, e.g., H. insolens as described in WO 96/13580, a Pseudomonas lipase, e.g., from P. alcaligenes or P. pseudoalcaligenes (EP 218 272), P. cepacia (EP 331 376), P. stutzeri (GB 1,372,034), P. fluorescens, Pseudomonas sp. strain SD 705 (WO 95/06720 and WO 96/27002), P. wisconsinensis (WO 96/12012), a Bacillus lipase, e.g., from B. subtilis (Dartois et al., 1993, Biochemica et Biophysica Acta, 1131: 253-360), B. stearothermophilus (JP 64/744992) or B. pumilus (WO 91/16422).

Other examples are lipase variants such as those described in WO 92/05249, WO 94/01541, EP 407 225, EP 260 105, WO 95/35381, WO 96/00292, WO 95/30744, WO 94/25578, WO 95/14783, WO 95/22615, WO 97/04079, WO 97/07202, WO 00/060063, WO 2007/087508 and WO 2009/109500.

Preferred commercially available lipase enzymes include Lipo Lipolase Ultra™, and Lipex™; Lecitase™, Lipolex™; Lipoclean™, Lipoprime™ (Novozymes A/S). Other commercially available lipases include Lumafast (DuPont); Lipomax (Gist-Brocades/DuPont) and Bacillus sp. lipase from Solvay.

Amylases

Suitable amylases (a and/or 3) include those of bacterial or fungal origin. Chemically modified or protein engineered mutants are included. Amylases include, for example, a amylases obtained from Bacillus, e.g., a special strain of Bacillus licheniformis, described in more detail in GB 1,296,839.

Examples of suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193. Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

### M197T;

### H156Y+A181T+N190F+A209V+Q264S; or

### G48A+T491+G107A+H156Y+A181T+N190F+1201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, I206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476. More preferred variants are those having a deletion in positions 181 and 182 or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

### N128C+K178L+T182G+Y305R+G475K;

### N128C+K178L+T182G+F202Y+Y305R+D319T+G475K;

### S125A+N128C+K178L+T182G+Y305R+G475K; or

S125A+N128C+T131I+T165I+K178L+T182G+Y305R+G475K wherein the variants are C-terminally truncated and optionally further comprises a substitution at position 243 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Stainzyme™ Stainzyme Plus™; Duramyl™, Termamyl™, Termamyl Ultra™; Natalase™, Fungamyl™ and BAN™ (Novozymes A/S), Rapidase™ and Purastar™/Effectenz™, Powerase™, Amplify™, Amplify Prime™, Preferenz™ S100, and Preferenz™ S110 (DuPont).

Deoxyribonuclease (DNase)

Suitable deoxyribonucleases (DNases) are any enzyme that catalyzes the hydrolytic cleavage of phosphodiester linkages in the DNA backbone, thus degrading DNA. According to the invention, a DNase which is obtainable from a bacterium is preferred; in particular a DNase which is obtainable from a Bacillus is preferred; in particular a DNase which is obtainable from Bacillus subtilis or Bacillus licheniformis is preferred. Examples of such DNases are described in patent application WO 2011/098579 or in PCT/EP2013/075922.

Perhydrolases

Suitable perhydrolases are capable of catalyzing a perhydrolysis reaction that results in the production of a peracid from a carboxylic acid ester (acyl) substrate in the presence of a source of peroxygen (e.g., hydrogen peroxide). While many enzymes perform this reaction at low levels, perhydrolases exhibit a high perhydrolysis:hydrolysis ratio, often greater than 1. Suitable perhydrolases may be of plant, bacterial or fungal origin. Chemically modified or protein engineered mutants are included.

Examples of useful perhydrolases include naturally occurring Mycobacterium perhydrolase enzymes, or variants thereof. An exemplary enzyme is derived from Mycobacterium smegmatis. Such enzyme, its enzymatic properties, its structure, and variants thereof, are described in WO 2005/056782, WO 2008/063400, US 2008/145353, and US2007167344.

Oxidases/Peroxidases

Suitable oxidases and peroxidases (or oxidoreductases) include various sugar oxidases, laccases, peroxidases and haloperoxidases.

Suitable peroxidases include those comprised by the enzyme classification EC 1.11.1.7, as set out by the Nomenclature Committee of the International Union of Biochemistry and Molecular Biology (IUBMB), or any fragment derived therefrom, exhibiting peroxidase activity.

Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257.

A peroxidase for use in the invention also include a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions.

In an embodiment, the haloperoxidase is a chloroperoxidase. Preferably, the haloperoxidase is a vanadium haloperoxidase, i.e., a vanadate-containing haloperoxidase. In a preferred method of the present invention the vanadate-containing haloperoxidase is combined with a source of chloride ion.

Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis. Haloperoxidases have also been isolated from bacteria such as Pseudomonas, e.g., P. pyrrocinia and Streptomyces, e.g., S. aureofaciens.

In an preferred embodiment, the haloperoxidase is derivable from Curvularia sp., in particular Curvularia verruculosa or Curvularia inaequalis, such as C. inaequalis CBS 102.42 as described in WO 95/27046; or C. verruculosa CBS 147.63 or C. verruculosa CBS 444.70 as described in WO 97/04102; or from Drechslera hartlebii as described in WO 01/79459, Dendryphiella salina as described in WO 01/79458, Phaeotrichoconis crotalarie as described in WO 01/79461, or Geniculosporium sp. as described in WO 01/79460.

An oxidase according to the invention include, in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5).

Preferred laccase enzymes are enzymes of microbial origin. The enzymes may be derived from plants, bacteria or fungi (including filamentous fungi and yeasts).

Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885).

Suitable examples from bacteria include a laccase derivable from a strain of Bacillus.

A laccase derived from Coprinopsis or Myceliophthora is preferred; in particular a laccase derived from Coprinopsis cinerea, as disclosed in WO 97/08325; or from Myceliophthora thermophila, as disclosed in WO 95/33836.

Examples of other oxidases include, but are not limited to, amino acid oxidase, glucose oxidase, lactate oxidase, galactose oxidase, polyol oxidase (e.g., WO2008/051491), and aldose oxidase. Oxidases and their corresponding substrates may be used as hydrogen peroxide generating enzyme systems, and thus a source of hydrogen peroxide. Several enzymes, such as peroxidases, haloperoxidases and perhydrolases, require a source of hydrogen peroxide. By studying EC 1.1.3._, EC 1.2.3._, EC 1.4.3._, and EC 1.5.3._ or similar classes (under the International Union of Biochemistry), other examples of such combinations of oxidases and substrates are easily recognized by one skilled in the art.

Amino acid changes, as referenced above, may be of a minor nature, that is conservative amino acid substitutions or insertions that do not significantly affect the folding and/or activity of the protein; small deletions, typically of 1-30 amino acids; small amino or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for enzyme activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46: 145; Ner et al., 1988, DNA 7: 127).

The relatedness between two amino acid sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues×100)/(Length of Alignment−Total Number of Gaps in Alignment).

Stabilizers/Inhibitors

Such enzyme(s), as described above, may be stabilized using conventional stabilizing agents, e.g., a polyol such as (mono, di or tri) propylene glycol, polyethylene glycol (such as PEG200-PEG1000), glycerol, a sugar or sugar alcohol(s); or compounds that act by temporarily reducing the activity of proteases.

Thus, the composition of the invention may also include a protease inhibitor/stabilizer, which is a reversible inhibitor of protease activity, e.g., serine protease activity. Preferably, the protease inhibitor is a (reversible) subtilisin protease inhibitor. In particular, the protease inhibitor may be a peptide aldehyde, boric acid, or a boronic acid; or a derivative of any of these.

The protease inhibitor may have an inhibition constant to a serine protease, Ki (mol/L) of from 1E-12 to 1E-03; more preferred from 1E-11 to 1E-04; even more preferred from 1E-10 to 1E-05; even more preferred from 1E-10 to 1E-06; and most preferred from 1E-09 to 1E-07.

Boronic Acid

The protease inhibitor may be boronic acid or a derivative thereof; preferably, phenylboronic acid or a derivative thereof. In an embodiment of the invention, the phenyl boronic acid derivative is of the following formula:

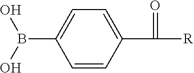

wherein R is selected from the group consisting of hydrogen, hydroxy, C1-C6 alkyl, substituted C1-C6 alkyl, C1-C6 alkenyl and substituted C1-C6 alkenyl. Preferably, R is hydrogen, CH3, CH3CH2 or CH3CH2CH2.

In a preferred embodiment, the protease inhibitor (phenyl boronic acid derivative) is 4-formyl-phenyl-boronic acid (4-FPBA).

In another particular embodiment, the protease inhibitor is selected from the group consisting of: thiophene-2 boronic acid, thiophene-3 boronic acid, acetamidophenyl boronic acid, benzofuran-2 boronic acid, naphtalene-1 boronic acid, naphtalene-2 boronic acid, 2-FPBA, 3-FBPA, 4-FPBA, 1-thianthrene boronic acid, 4-dibenzofuran boronic acid, 5-methylthiophene-2 boronic, acid, thionaphtrene boronic acid, furan-2 boronic acid, furan-3 boronic acid, 4,4 biphenyl-diborinic acid, 6-hydroxy-2-naphtalene, 4-(methylthio) phenyl boronic acid, 4 (trimethyl-silyl)phenyl boronic acid, 3-bromothiophene boronic acid, 4-methylthiophene boronic acid, 2-naphtyl boronic acid, 5-bromothiphene boronic acid, 5-chlorothiophene boronic acid, dimethylthiophene boronic acid, 2-bromophenyl boronic acid, 3-chlorophenyl boronic acid, 3-methoxy-2-thiophene, p-methyl-phenylethyl boronic acid, 2-thianthrene boronic acid, di-benzothiophene boronic acid, 4-carboxyphenyl boronic acid, 9-anthryl boronic acid, 3,5 dichlorophenyl boronic, acid, diphenyl boronic acidanhydride, o-chlorophenyl boronic acid, p-chlorophenyl boronic acid, m-bromophenyl boronic acid, p-bromophenyl boronic acid, p-flourophenyl boronic acid, p-tolyl boronic acid, o-tolyl boronic acid, octyl boronic acid, 1,3,5 trimethylphenyl boronic acid, 3-chloro-4-flourophenyl boronic acid, 3-aminophenyl boronic acid, 3,5-bis-(triflouromethyl) phenyl boronic acid, 2,4 dichlorophenyl boronic acid, 4-methoxyphenyl boronic acid.

Further boronic acid derivatives suitable as protease inhibitors in the detergent composition are described in U.S. Pat. Nos. 4,963,655, 5,159,060, WO 95/12655, WO 95/29223, WO 92/19707, WO 94/04653, WO 94/04654, U.S. Pat. Nos. 5,442,100, 5,488,157 and 5,472,628.

Peptide Aldehyde or Ketone

The protease stabilizer may have the formula: P-(A)y-L-(B)x-B0-R* wherein:
R* is H (hydrogen), CH3, CX3, CHX2, or CH2X. Preferably, R*═H so that the stabilizer is a peptide aldehyde with the formula P-(A)y-L-(B)x-B0-H;
X is a halogen atom, particularly F (fluorine);
B0 is a single amino acid residue with L- or D-configuration of the formula —NH—CH(R)—C(═O)—;
x is 1, 2 or 3;
Bx is independently a single amino acid residue, each connected to the next B or to B0 via its C-terminal;
L is absent or independently a linker group of the formula —C(═O)—, —C(═O)—C(═O)—, —C(═S)—, —C(═S)—C(═S)— or —C(═S)—C(═O)—;
A is absent if L is absent or is independently a single amino acid residue connected to L via the N-terminal of the amino acid;
P is selected from the group consisting of hydrogen or if L is absent an N-terminal protection group;
y is 0, 1, or 2,
R is independently selected from the group consisting of C1-6 alkyl, C6-10 aryl or C7-10 arylalkyl optionally substituted with one or more, identical or different, substituent's R′;
R′ is independently selected from the group consisting of halogen, —OH, —OR″, —SH, —SR″, —NH2, —NHR″, —NR″2, —CO2H, —CONH2, —CONHR″, —CONR″2, —NHC(═N)NH2; and
R″ is a C1-6 alkyl group.
x may be 1, 2 or 3 and therefore B may be 1, 2 or 3 amino acid residues respectively.

Thus, B may represent B1, B2-B1 or B3-B2-B1, where B3, B2 and B1 each represent one amino acid residue. y may be 0, 1 or 2 and therefore A may be absent, or 1 or 2 amino acid residues respectively having the formula A1 or A2-A1 wherein A2 and A1 each represent one amino acid residue.

B0 may be a single amino acid residue with L- or D-configuration, which is connected to H via the C-terminal of the amino acid. B0 has the formula —NH—CH(R)—C(═O)—, wherein R is a C1-6 alkyl, C6-10 aryl or C7-10 arylalkyl side chain, such as methyl, ethyl, propyl, isopropyl, butyl, isobutyl, phenyl or benzyl, and wherein R may be optionally substituted with one or more, identical or different, substituent's R′. Particular examples of B0 are the D- or L-form of arginine (Arg), 3,4-dihydroxyphenylalanine, isoleucine (Ile), leucine (Leu), methionine (Met), norleucine (Nle), norvaline (Nva), phenylalanine (Phe), m-tyrosine, p-tyrosine (Tyr) and valine (Val). A particular embodiment is when B0 is leucine, methionine, phenylalanine, p-tyrosine and valine.

B1, which is connected to B0 via the C-terminal of the amino acid, may be an aliphatic, hydrophobic and/or neutral amino acid. Examples of B1 are alanine (Ala), cysteine (Cys), glycine (Gly), isoleucine (Ile), leucine (Leu), norleucine (Nle), norvaline (Nva), proline (Pro), serine (Ser), threonine (Thr) and valine (Val). Particular examples of B1 are alanine, glycine, isoleucine, leucine and valine. A particular embodiment is when B1 is alanine, glycine or valine.

If present, B2, which is connected to B1 via the C-terminal of the amino acid, may be an aliphatic, hydrophobic, neutral and/or polar amino acid. Examples of B2 are alanine (Ala), arginine (Arg), capreomycidine (Cpd), cysteine (Cys), glycine (Gly), isoleucine (Ile), leucine (Leu), norleucine (Nie), norvaline (Nva), phenylalanine (Phe), proline (Pro), serine (Ser), threonine (Thr), and valine (Val). Particular examples of B2 are alanine, arginine, capreomycidine, glycine, isoleucine, leucine, phenylalanine and valine. A particular embodiment is when B2 is arginine, glycine, leucine, phenylalanine or valine.

B3, which if present is connected to B2 via the C-terminal of the amino acid, may be a large, aliphatic, aromatic, hydrophobic and/or neutral amino acid. Examples of B3 are isoleucine (Ile), leucine (Leu), norleucine (Nle), norvaline (Nva), phenylalanine (Phe), phenylglycine, tyrosine (Tyr), tryptophan (Trp) and valine (Val). Particular examples of B3 are leucine, phenylalanine, tyrosine and tryptophan.

The linker group L may be absent or selected from the group consisting of —C(═O)—, —C(═O)—C(═O)—, —C(═S)—, —C(═S)—C(═S)— or —C(═S)—C(═O)—. Particular embodiments of the invention are when L is absent or L is a carbonyl group —C(═O)—.

A1, which if present is connected to L via the N-terminal of the amino acid, may be an aliphatic, aromatic, hydrophobic, neutral and/or polar amino acid. Examples of A1 are alanine (Ala), arginine (Arg), capreomycidine (Cpd), glycine (Gly), isoleucine (Ile), leucine (Leu), norleucine (Nle), norvaline (Nva), phenylalanine (Phe), threonine (Thr), tyrosine (Tyr), tryptophan (Trp) and valine (Val). Particular examples of A1 are alanine, arginine, glycine, leucine, phenylalanine, tyrosine, tryptophan and valine. A particular embodiment is when B2 is leucine, phenylalanine, tyrosine or tryptophan.

The A2 residue, which if present is connected to A1 via the N-terminal of the amino acid, may be a large, aliphatic, aromatic, hydrophobic and/or neutral amino acid. Examples of A2 are arginine (Arg), isoleucine (Ile), leucine (Leu), norleucine (Nle), norvaline (Nva), phenylalanine (Phe), phenylglycine, Tyrosine (Tyr), tryptophan (Trp) and valine (Val). Particular examples of A2 are phenylalanine and tyrosine.

The N-terminal protection group P (if present) may be selected from formyl, acetyl (Ac), benzoyl (Bz), trifluoroacetyl, methoxysuccinyl, aromatic and aliphatic urethane protecting groups such as fluorenylmethyloxycarbonyl (Fmoc), methoxycarbonyl (Moc), (fluoromethoxy)carbonyl, benzyloxycarbonyl (Cbz), t-butyloxycarbonyl (Boc) and adamantyloxycarbonyl; p-methoxybenzyl carbonyl, benzyl (Bn), p-methoxybenzyl (PMB), p-methoxyphenyl (PMP), methoxyacetyl, methylamino carbonyl, methylsulfonyl, ethylsulfonyl, benzylsulfonyl, methylphosphoramidyl (MeOP(OH)(═O)) and benzylphosphoramidyl (PhCH2OP(OH)(═O)).

In the case of a tripeptide aldehyde with a protection group (i.e. x=2, L is absent and A is absent), P is preferably acetyl, methoxycarbonyl, benzyloxycarbonyl, methylamino carbonyl, methylsulfonyl, benzylsulfonyl and benzylphosphoramidyl. In the case of a tetrapeptide aldehyde with a protection group (i.e. x=3, L is absent and A is absent), P is preferably acetyl, methoxycarbonyl, methylsulfonyl, ethylsulfonyl and methylphosphoramidyl.

Suitable peptide aldehydes are described in WO94/04651, WO95/25791, WO98/13458, WO98/13459, WO98/13460, WO98/13461, WO98/13462, WO07/141736, WO07/145963, WO09/118375, WO10/055052 and WO11/036153. More particularly, the peptide aldehyde may be Cbz-Arg-Ala-Tyr-H, Ac-Gly-Ala-Tyr-H, Cbz-Gly-Ala-Tyr-H, Cbz-Gly-Ala-Tyr-CF3, Cbz-Gly-Ala-Leu-H, Cbz-Val-Ala-Leu-H, Cbz-Val-Ala-Leu-CF3, Moc-Val-Ala-Leu-CF3, Cbz-Gly-Ala-Phe-H, Cbz-Gly-Ala-Phe-CF3, Cbz-Gly-Ala-Val-H, Cbz-Gly-Gly-Tyr-H, Cbz-Gly-Gly-Phe-H, Cbz-Arg-Val-Tyr-H, Cbz-Leu-Val-Tyr-H, Ac-Leu-Gly-Ala-Tyr-H, Ac-Phe-Gly-Ala-Tyr-H, Ac-Tyr-Gly-Ala-Tyr-H, Ac-Phe-Gly-Ala-Leu-H, Ac-Phe-Gly-Ala-Phe-H, Ac-Phe-Gly-Val-Tyr-H, Ac-Phe-Gly-Ala-Met-H, Ac-Trp-Leu-Val-Tyr-H, MeO—CO-Val-Ala-Leu-H, MeNCO-Val-Ala-Leu-H, MeO—CO-Phe-Gly-Ala-Leu-H, MeO—CO-Phe-Gly-Ala-Phe-H, MeSO2-Phe-Gly-Ala-Leu-H, MeSO2-Val-Ala-Leu-H, PhCH2O—P(OH)(O)-Val-Ala-Leu-H, EtSO2-Phe-Gly-Ala-Leu-H, PhCH2SO2-Val-Ala-Leu-H, PhCH2O—P(OH)(O)-Leu-Ala-Leu-H, PhCH2O—P(OH)(O)-Phe-Ala-Leu-H, or MeO—P(OH)(O)-Leu-Gly-Ala-Leu-H. A preferred stabilizer for use in the liquid composition of the invention is Cbz-Gly-Ala-Tyr-H, or a hydrosulfite adduct thereof, wherein Cbz is benzyloxycarbonyl.

Further examples of such peptide aldehydes include α-MAPI, β-MAPI, Phe-C(═O)-Arg-Val-Tyr-H, Phe-C(═O)-Gly-Gly-Tyr-H, Phe-C(═O)-Gly-Ala-Phe-H, Phe-C(═O)-Gly-Ala-Tyr-H, Phe-C(═O)-Gly-Ala-L-H, Phe-C(═O)-Gly-Ala-Nva-H, Phe-C(═O)-Gly-Ala-Nle-H, Tyr-C(═O)-Arg-Val-Tyr-H, Tyr-C(═O)-Gly-Ala-Tyr-H, Phe-C(═S)-Arg-Val-Phe-H, Phe-C(═S)-Arg-Val-Tyr-H, Phe-C(═S)-Gly-Ala-Tyr-H, Antipain, GE20372A, GE20372B, Chymostatin A, Chymostatin B, and Chymostatin C.

Hydrosulfite Adduct

The protease stabilizer may be a hydrosulfite adduct of the peptide aldehyde described above, e.g. as described in WO 2013/004636. The adduct may have the formula P-(A)y-L-(B)x-N(H)—CHR—CH(OH)-503M, wherein P, A, y, L, B, x and R are defined as above, and M is H or an alkali metal, preferably Na or K. A preferred embodiment is a hydrosulfite adduct wherein P=Cbz, B2=Gly; B1=Ala; B0=Tyr (so R=PhCH2, R′═OH), x=2, y=0, L=A=absent and M=Na.

Peptide Aldehyde or Hydrosulfite Adduct

The protease stabilizer may be an aldehyde having the formula P-B2-B1-B0-H or an adduct having the formula P-B2-B1-N(H)—CHR—CHOH—SO3M (see also WO 2013/004636), wherein

a) H is hydrogen;
b) B0 is a single amino acid residue with L- or D-configuration of the formula —NH—CH(R)—C(═O)—;
c) B1 and B2 are independently single amino acid residues;
d) R is independently selected from the group consisting of C1-6 alkyl, C6-10 aryl or C7-10 arylalkyl optionally substituted with one or more, identical or different, substituent's R′;
e) R′ is independently selected from the group consisting of halogen, —OH, —OR″, —SH, —SR″, —NH2, —NHR″, —NR″2, —CO2H, —CONH2, —CONHR″, —CONR″2, —NHC(═N)NH2;
f) R″ is a C1-6 alkyl group; and
g) P is an N-terminal protection group.

Constituents b) to g) may be selected as described above.

An aqueous solution of the hydrosulfite adduct may be prepared by reacting the corresponding peptide aldehyde with an aqueous solution of sodium bisulfite (sodium hydrogen sulfite, NaHSO3); potassium bisulfite (KHSO3) by known methods, e.g., as described in WO 98/47523; U.S. Pat. Nos. 6,500,802; 5,436,229; J. Am. Chem. Soc. (1978) 100, 1228; Org. Synth., Coll. vol. 7: 361.

The molar ratio of the above-mentioned peptide aldehydes (or hydrosulfite adducts) to the protease may be at least 1:1 or 1.5:1, and it may be less than 1000:1, more preferred less than 500:1, even more preferred from 100:1 to 2:1 or from 20:1 to 2:1, or most preferred, the molar ratio is from 10:1 to 2:1.

Formate salts (e.g., sodium formate) and formic acid have also shown good effects as inhibitor of protease activity. Formate can be used synergistically with the above-mentioned protease inhibitors, as shown in WO 2013/004635. The formate salts may be present in the pouch composition in an amount of at least 0.1% w/w or 0.5% w/w, e.g., at least 1.0%, at least 1.2% or at least 1.5%. The amount is typically below 5% w/w, below 4% or below 3%.

In an embodiment, the protease is a metalloprotease and the inhibitor is a metalloprotease inhibitor, e.g., a protein hydrolysate based inhibitor (e.g., as described in WO 2008/134343).

The material in the pouch may also contain enzyme inhibitors and other materials which provide stability to the enzyme themselves. Preferred additives include Z-GAY-H, and the bisulfite form thereof; and 4-FPBA. Z-GAY-H is Cbz-Gly-Ala-NHCH(CH₂C₆H₄pOH)CHO, wherein Cbz is benzyloxycarbonyl. 4-FPBA is 4-formyl phenyl boronic acid.

Various different types of enzymes may be used in the invention, such as those which are traditionally used in detergent compositions. These may include proteases, lipases, cellulases, and/or amylases. The pouch may contain one form of enzyme, or may contain more than one type of enzyme in the same pouch.

The enzyme is preferably spray-dried. To prepare the pouches, an enzyme in solid form is provided. This may be prepared by spray drying a liquid form of the enzyme, such as a solution of the enzyme. The solution may contain components other than the enzyme. Another method is to spray dry a solution of the enzyme, and then so add one or more additives to the spray dried solid. This process provides a spray dried powder which can be mixed with any desired additives and then placed in a pouch. The pouch may be pre-formed or may be formed in a continuous form, fill and seal process to form the pouch containing the enzyme-containing material. Once formed, the pouch provides a stable and pre-measured form of the enzyme, which can then be used in the preparation of enzyme-containing compositions, such as enzyme-containing detergent compositions.

To form the enzyme-containing detergent composition, a pouch may be added to an aqueous composition, such as an aqueous detergent composition. The pouch will then dissolve to release the contents, allowing the enzyme to be spread throughout the final composition. Dissolution can be achieved at any desired temperature, such as at room temperature or at an elevated temperature such as at about 40° C. or about 60° C. Agitation, preferably by stirring, provides an efficient way to aid the process. Ideally, the dissolution time should be relatively small such as from a few minutes to a few hours. Ideally the pouch will dissolve in less than an hour, preferably less than 30 minutes, and more preferably less than 15 minutes. Such a relatively short timescale would allow for use in traditional methods of preparing detergent composition. The resulting enzyme-containing composition can then be processed in the normal way, for example by being packaged into containers ready for sale and else.

The enzyme-containing material may comprise one or more additives to provide various functions or benefits. For example, the enzyme-containing material may comprise a surfactant, which may help in the dissolution of the enzyme into the detergent composition. The surfactant may be a non-ionic surfactant, preferably a polyoxyethylene alkyl ether surfactant. The polyoxyethylene alkyl ether surfactant is preferably a Softanol™ surfactant (available from Ineos Oxide), preferably Softanol™ 90.

The content of the pouch may contain a polyol, such as glycerol, (mono, di, or tri) propylene glycol, sugar alcohol (e.g., sorbitol), a polypropylene glycol (PPG), or a polyethylene glycol (PEG). Polyethylene glycol with a molecular weight in the range of 200-1000 are preferred polyols for use in the invention, with PEG300 and PEG400 being particularly preferred. These materials have been found to provide benefits including helping the dissolution of the enzyme into the detergent composition.

Preferably, the composition contains from 0.5% to 90% w/w active enzyme protein, more preferably 1% to 90% w/w, more preferably 5% to 90% w/w, more preferably 5% to 50% w/w active enzyme protein.

Microorganism(s)

The microorganisms may be one or more fungi, yeast, or bacteria; such as dehydrated bacteria or yeast.

In an embodiment, the microorganism is a microbial spore (as opposed to vegetative cells), such as bacterial (endo)spores; or fungal spores, conidia, hypha.

Particularly preferred are Bacillus endospores, such as endospores of Bacillus subtilis, Bacillus licheniformis, Bacillus amyloliquefaciens, and/or Bacillus megaterium.

Detergent Composition

The enzyme-containing pouch of the invention may be added to, and thus form part of, any detergent composition in any form, such as liquid detergents, and soap and detergent bars (e.g., syndet bars). After addition of the enzyme pouch of the invention, the detergent composition comprises the water-soluble polymer of the wall of the pouch, such as poly(vinyl alcohol) (PVA).

The enzyme-containing pouch, as described above, may be added to the liquid detergent composition in an amount corresponding to from 0.0001% to 5% (w/w) active enzyme protein (AEP); preferably from 0.0005% to 5%, more preferably from 0.0005% to 2%, more preferably from 0.0005% to 1%, even more preferably from 0.001% to 1%, and most preferably from 0.005% to 1% (w/w) active enzyme protein.

The liquid detergent composition has a physical form, which is not solid (or gas). It may be a pourable liquid, a paste, a pourable gel or a non-pourable gel. It may be either isotropic or structured, preferably isotropic. It may be a formulation useful for washing in automatic washing machines or for hand washing, or for (automatic) dish wash. It may also be a personal care product, such as a shampoo, toothpaste, or hand soap.

The liquid detergent composition is capable of dissolving the pouch of the invention and may be aqueous, typically containing at least 20% by weight and up to 95% water, such as up to 70% water, up to 50% water, up to 40% water, or up to 30% water. Other types of liquids, including without limitation, alkanols, amines, diols, ethers and polyols may be included in an aqueous liquid detergent. An aqueous liquid detergent may contain from 0-30% organic solvent.

The choice of detergent components may include, for textile care, the consideration of the type of textile to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

The choice of additional components is within the skill of the artisan and includes conventional ingredients, including the exemplary non-limiting components set forth below.

Surfactants

The detergent composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant(s) is chosen based on the desired cleaning application, and includes any conventional surfactant(s) known in the art. Any surfactant known in the art for use in detergents may be utilized.

When included therein the detergent will usually contain from about 1% to about 40% by weight, such as from about 5% to about 30%, including from about 5% to about 15%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or soap, and combinations thereof.

When included therein the detergent will usually contain from about 0.1% to about 10% by weight of a cationic surfactant. Non-limiting examples of cationic surfactants include alklydimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, and combinations thereof.

When included therein the detergent will usually contain from about 0.2% to about 40% by weight of a non-ionic surfactant, for example from about 0.5% to about 30%, in particular from about 1% to about 20%, from about 3% to about 10%, such as from about 3% to about 5%, or from about 8% to about 12%. Non-limiting examples of non-ionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxy alkyl fatty acid amides, or N-acyl N-alkyl derivatives of glucosamine (glucamides, GA, or fatty acid glucamide, FAGA), as well as products available under the trade names SPAN and TWEEN, and combinations thereof.

When included therein the detergent will usually contain from about 0.1% to about 20% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, fatty acid alkanolamides and ethoxylated fatty acid alkanolamides, and combinations thereof.

When included therein the detergent will usually contain from about 0.1% to about 10% by weight of a zwitterionic surfactant. Non-limiting examples of zwitterionic surfactants include betaine, alkyldimethylbetaine, sulfobetaine, and combinations thereof.

Hydrotropes

A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants); however the molecular structure of hydrotropes generally do not favor spontaneous self-aggregation, see for example review by Hodgdon and Kaler (2007), Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in detergent compositions allow for example more concentrated formulations of surfactants (as in the process of compacting liquid detergents by removing water) without inducing undesired phenomena such as phase separation or high viscosity.

The detergent may contain 0-5% by weight, such as about 0.5 to about 5%, or about 3% to about 5%, of a hydrotrope. Any hydrotrope known in the art for use in detergents may be utilized. Non-limiting examples of hydrotropes include sodium benzene sulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

Builders and Co-Builders

The detergent composition may contain about 0-65% by weight, such as about 5% to about 50% of a detergent builder or co-builder, or a mixture thereof. In a dish wash detergent, the level of builder is typically 40-65%, particularly 50-65%. The builder and/or co-builder may particularly be a chelating agent that forms water-soluble complexes with Ca and Mg ions. Any builder and/or co-builder known in the art for use in laundry detergents may be utilized. Non-limiting examples of builders include citrates, diphosphates (pyrophosphates), triphosphates such as sodium triphosphate (STP or STPP), carbonates such as sodium carbonate, ethanolamines such as 2-aminoethan-1-ol (MEA), diethanolamine (DEA, also known as iminodiethanol), triethanolamine (TEA, also known as 2,2′,2″-nitrilotriethanol), and carboxymethyl inulin (CMI), and combinations thereof.

The detergent composition may also contain 0-50% by weight, such as about 5% to about 30%, of a detergent co-builder, or a mixture thereof. The detergent composition may include include a co-builder alone, or in combination with a builder, for example a citrate builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N, N-diacetic acid (α-ALDA), serine-N, N-diacetic acid (SEDA), isoserine-N, N-diacetic acid (ISDA), phenylalanine-N, N-diacetic acid (PHDA), anthranilic acid-N, N-diacetic acid (ANDA), sulfanilic acid-N, N-diacetic acid (SLDA), taurine-N, N-diacetic acid (TUDA) and sulfomethyl-N, N-diacetic acid (SMDA), N-(2-hydroxyethyl)-ethylidenediamine-N, N′, N′-triacetate (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053.

Polymers

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575 and U.S. Pat. No. 5,955,415. Salts of the above-mentioned polymers are also contemplated.

Fabric Hueing Agents

The detergent compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO 2005/03274, WO 2005/03275, WO 2005/03276 and EP 1876226 (hereby incorporated by reference). The detergent composition preferably comprises from 0.0001 to 0.2% w/w fabric hueing agent. Suitable hueing agents are also disclosed in, e.g., WO 2007/087257 and WO 2007/087243.

Adjunct Materials

Any detergent components known in the art for use in laundry detergents may also be utilized. Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including the enzyme stabilizers/inhibitors mentioned above, CMC, and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in laundry detergents may be utilized. The choice of such ingredients is well within the skill of the artisan.

Dispersants—

The detergent compositions of the present invention can also contain dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker, Inc.

Dye Transfer Inhibiting Agents—

The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.0001% to about 10%, from about 0.01% to about 5% or even from about 0.1% to about 3% by weight of the composition.

Fluorescent Whitening Agent—

The detergent compositions of the present invention will preferably also contain additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2.2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins.

Suitable fluorescent brightener levels include lower levels of from about 0.01, from 0.05, from about 0.1 or even from about 0.2% w/w to upper levels of 0.5 or even 0.75% w/w.

Soil Release Polymers—

The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers are amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore random graft co-polymers are suitable soil release polymers. Suitable graft co-polymers are described in more detail in WO 2007/138054, WO 2006/108856 and WO 2006/113314 (hereby incorporated by reference). Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

Anti-Redeposition Agents—

The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

Rheology Modifiers are structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of the composition. The rheology and viscosity of the detergent can be modified and adjusted by methods known in the art, for example as shown in EP 2169040.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

Bleaching Systems

Examples of liquid detergents combining bleach and enzymes include, e.g., U.S. Pat. No. 5,275,753 and WO 99/00478. The detergent may contain 0-50% of a bleaching system. Any bleaching system known in the art for use in laundry detergents may be utilized. Suitable bleaching system components include bleaching catalysts such as MnTACN, photobleaches, bleach activators, sources of hydrogen peroxide such as sodium percarbonate and sodium perborates, preformed peracids and mixtures thereof.

Some embodiments of the invention include:

### Embodiment 1

A pouch comprising a wall forming a chamber, the wall comprising a water-soluble polymer, and the chamber containing a particulate solid comprising an enzyme or a microorganism, and a liquid comprising a surfactant and/or a polyol.

### Embodiment 2

The pouch according to embodiment 1, which weighs more than 50 g.

### Embodiment 3

The pouch according to embodiment 1, which weighs more than 100 g.

### Embodiment 4

The pouch according to embodiment 1, which weighs more than 250 g.

### Embodiment 5

The pouch according to embodiment 1, which weighs more than 500 g.

### Embodiment 6

The pouch according to any one of embodiments 1-5, which weighs less than 25 kg.

### Embodiment 7

The pouch according to any one of embodiments 1-5, which weighs less than 10 kg.

### Embodiment 8

The pouch according to any one of embodiments 1-5, which weighs less than 5000 g.

### Embodiment 9

The pouch according to any one of embodiments 1-5, which weighs less than 1000 g.

### Embodiment 10

The pouch according to any one of embodiments 1-9, wherein the content of the chamber contains at least 10% w/w of the particulate solid.

### Embodiment 11

The pouch according to any one of embodiments 1-9, wherein the content of the chamber contains at least 20% w/w of the particulate solid.

### Embodiment 12

The pouch according to any one of embodiments 1-9, wherein the content of the chamber contains at least 30% w/w of the particulate solid.

### Embodiment 13

The pouch according to any one of embodiments 1-9, wherein the content of the chamber contains at least 40% w/w of the particulate solid.

### Embodiment 14

The pouch according to any one of embodiments 1-9, wherein the content of the chamber contains at least 50% w/w of the particulate solid.

### Embodiment 15

The pouch according to any one of embodiments 1-14, which comprises more than 1% w/w active enzyme protein.

### Embodiment 16

The pouch according to any one of embodiments 1-14, which comprises more than 5% w/w active enzyme protein.

### Embodiment 17

The pouch according to any one of embodiments 1-14, which comprises more than 10% w/w active enzyme protein.

### Embodiment 18

The pouch according to any one of embodiments 1-17, which comprises less than 90% w/w active enzyme protein.

### Embodiment 19

The pouch according to any one of embodiments 1-17, which comprises less than 50% w/w active enzyme protein.

### Embodiment 20

The pouch according to any one of embodiments 1-19, wherein the enzyme is selected from a protease (e.g., subtilisin or metalloprotease), lipase, cutinase, amylase, carbohydrase, cellulase, pectinase, mannanase, arabinase, galactanase, xanthanase, xylanase, DNase, perhydrolase, oxidoreductase (e.g., laccase, peroxidase, peroxygenase and/or haloperoxidase).

### Embodiment 21

The pouch according to any one of embodiments 1-19, wherein the enzyme is selected from a protease, lipase, amylase, cellulase, pectinase, mannanase, and/or DNase.

### Embodiment 22

The pouch according to any one of embodiments 1-19, wherein the enzyme is a protease, such as a subtilisin.

### Embodiment 23

The pouch according to any one of embodiments 1-22, wherein the solid comprises a spray dried enzyme.

### Embodiment 24

The pouch according to any one of embodiments 1-14, wherein the microorganism is a bacterium, fungus or yeast.

### Embodiment 25

The pouch according to any one of embodiments 1-14, wherein the microorganism is a dehydrated bacterium or yeast.

### Embodiment 26

The pouch according to any one of embodiments 1-14, wherein the microorganism is a microbial spore.

### Embodiment 27

The pouch according to any one of embodiments 1-14, wherein the microorganism is a bacterial (endo)spore.

### Embodiment 28

The pouch according to any one of embodiments 1-14, wherein the microorganism is a Bacillus endospore.

### Embodiment 29

The pouch according to any one of embodiments 1-14, wherein the microorganism is an endospore of Bacillus subtilis, Bacillus licheniformis, Bacillus amyloliquefaciens, or Bacillus megaterium.

### Embodiment 30

The pouch according to any one of embodiments 1-29, wherein the wall comprises PVA.

### Embodiment 31

The pouch according to embodiment 30, wherein the degree of hydrolysis of the PVA is from about 75% to about 99%.

### Embodiment 32

The pouch according to any one of embodiments 1-31, wherein the wall thickness is more than 20 μm.

### Embodiment 33

The pouch according to any one of embodiments 1-31, wherein the wall thickness is more than 50 μm.

### Embodiment 34

The pouch according to any one of embodiments 1-31, wherein the wall thickness is more than 100 μm.

### Embodiment 35

The pouch according to any one of embodiments 1-34, wherein the wall thickness is less than 5000 μm.

### Embodiment 36

The pouch according to any one of embodiments 1-34, wherein the wall thickness is less than 3000 μm.

### Embodiment 37

The pouch according to any one of embodiments 1-34, wherein the wall thickness is less than 2000 μm.

### Embodiment 38

The pouch according to any one of embodiments 1-34, wherein the wall thickness is less than 1000 μm.

### Embodiment 39

The pouch according to any one of embodiments 1-38, which comprises more than one enzyme.

### Embodiment 40

The pouch according to any one of embodiments 1-39, which comprises more than one chamber.

### Embodiment 41

The pouch according to any one of embodiments 1-40, wherein the surfactant comprises a non-ionic surfactant.

### Embodiment 42

The pouch according to any one of embodiments 1-41, wherein the surfactant comprises a polyoxyethylene alkyl ether surfactant or an alcohol ethoxylate surfactant.

### Embodiment 43

The pouch according to any one of embodiments 1-42, wherein the surfactant is a Softanol™ surfactant.

### Embodiment 44

The pouch according to any one of embodiments 1-43, wherein the polyol comprises glycerol.

### Embodiment 45

The pouch according to any one of embodiments 1-44, wherein the polyol comprises (mono, di, or tri) propylene glycol.

### Embodiment 46

The pouch according to any one of embodiments 1-45, wherein the polyol comprises a sugar alcohol, such as sorbitol.

### Embodiment 47

The pouch according to any one of embodiments 1-46, wherein the polyol comprises polyethylene glycol, preferably polyethylene glycol with a molecular weight in the range of 200-800.

### Embodiment 48

The pouch according to any one of embodiments 1-47, wherein the polyol comprises polypropylene glycol, preferably polypropylene glycol with a molecular weight in the range of 200-800.

### Embodiment 49

The pouch according to any one of embodiments 1-48, wherein the chamber further contains one or more additive.

### Embodiment 50

The pouch according to embodiment 49, wherein the one or more additive comprises an enzyme inhibitor.

### Embodiment 51

The pouch according to embodiment 50, wherein the enzyme is a protease and the enzyme inhibitor is a peptide aldehyde protease inhibitor, such as Z-GAY-H.

### Embodiment 52

The pouch according to embodiment 50, wherein the enzyme is a protease and the enzyme inhibitor is a boronic acid protease inhibitor, such as 4-FPBA.

### Embodiment 53

The pouch according to embodiment 50 or 51, wherein the additive comprises a protease inhibitor, the protease inhibitor having the formula P-(A)y-L-Bx-B0-R* or a hydrosulfite adduct thereof, wherein:

a) R* is H (hydrogen), CH3, CX3, CHX2, or CH2X;
b) X is a halogen atom;
c) B0 is a single amino acid residue of the formula —NH—CH(R)—C(═O)—;
d) x is 1, 2 or 3;
e) Bx (B1, B2, B3) is independently a single amino acid residue, each connected to the next B or to B0 via its C-terminal;
f) L is absent or independently a linker group of the formula —C(═O)—, —C(═O)—C(═O)—, —C(═S)—, —C(═S)—C(═S)— or —C(═S)—C(═O)—;
g) A is absent if L is absent or is independently a single amino acid residue connected to L via the N-terminal of the amino acid;
h) P is selected from the group consisting of hydrogen or if L is absent an N-terminal protection group;
i) y is 0, 1, or 2,
j) R is independently selected from the group consisting of C1-6 alkyl, C6-10 aryl or C7-10 arylalkyl optionally substituted with one or more, identical or different, substituent's R′;
k) R′ is independently selected from the group consisting of halogen, —OH, —OR″, —SH, —SR″, —NH2, —NHR″, —NR″2, —CO2H, —CONH2, —CONHR″, —CONR″2, —NHC(═N)NH2; and
l) R″ is a C1-6 alkyl group.
m) x may be 1, 2 or 3.

### Embodiment 54

The pouch according to embodiment 53, wherein x=2, L is absent, A is absent, and P is p-methoxycarbonyl (Moc) or benzyloxycarbonyl (Cbz).

### Embodiment 55

The pouch according to embodiment 50 or 51, wherein the enzyme inhibitor is a peptide aldehyde having the formula P-B3-B2-B1-B0-H or a hydrosulfite adduct having the formula P-B3-B2-B1-N(H)—CHR—CHOH—SO₃M, wherein

a) H is hydrogen;
b) B0 is a single amino acid residue of the formula —NH—CH(R)—C(═O)—;
c) B1 and B2 are independently single amino acid residues;
d) B3 is a single amino acid residue, or is absent;
d) R is independently selected from the group consisting of C1-6 alkyl, C6-10 aryl or C7-10 arylalkyl optionally substituted with one or more, identical or different, substituent's R′;
e) R′ is independently selected from the group consisting of halogen, —OH, —OR″, —SH, —SR″, —NH2, —NHR″, —NR″2, —CO2H, —CONH2, —CONHR″, —CONR″2, —NHC(═N)NH2;
f) R″ is a C1-6 alkyl group;
g) P is an N-terminal protection group; and
h) M is H or an alkali metal, preferably Na or K.

### Embodiment 56

The pouch according to embodiment 50 or 51, wherein the enzyme inhibitor is a peptide aldehyde having the formula P-B2-B1-B0-H or a hydrosulfite adduct having the formula P-B2-B1-N(H)—CHR—CHOH—SO₃M, wherein

a) H is hydrogen;
b) B0 is a single amino acid residue of the formula —NH—CH(R)—C(═O)—;
c) B1 and B2 are independently single amino acid residues;
d) R is independently selected from the group consisting of C1-6 alkyl, C6-10 aryl or C7-10 arylalkyl optionally substituted with one or more, identical or different, substituent's R′;
e) R′ is independently selected from the group consisting of halogen, —OH, —OR″, —SH, —SR″, —NH2, —NHR″, —NR″2, —CO2H, —CONH2, —CONHR″, —CONR″2, —NHC(═N)NH2;
f) R″ is a C1-6 alkyl group;
g) P is an N-terminal protection group; and
h) M is H or an alkali metal, preferably Na or K.

### Embodiment 57

The pouch according to embodiment 50 or 51, wherein the enzyme inhibitor is a peptide aldehyde having the formula P-B2-B1-B0-H or a hydrosulfite adduct having the formula P-B2-B1-B0-SO₃M, wherein

a) H is hydrogen;

### b) B0 is Phe, Tyr or Leu;

c) B1 and B2 are independently single amino acid residues;
d) P is an N-terminal protection group; and
e) M is H or an alkali metal, preferably Na or K.

### Embodiment 58

The pouch according to any one of embodiments 53-55, wherein B3 is leucine, phenylalanine, tyrosine, or tryptophan.

### Embodiment 59

The pouch according to embodiment 58, wherein B3 is leucine, phenylalanine, or tyrosine.

### Embodiment 60

The pouch according to any one of embodiments 53-56, wherein B0 is leucine, methionine, phenylalanine, p-tyrosine, or valine.

### Embodiment 61

The pouch according to embodiment 60, wherein B0 is leucine, phenylalanine, or p-tyrosine.

### Embodiment 62

The pouch according to any one of embodiments 53-61, wherein B1 is alanine, glycine, or valine.

### Embodiment 63

The pouch according to any one of embodiments 53-62, wherein B2 is arginine, glycine, leucine, phenylalanine, or valine.

### Embodiment 64

The pouch according to embodiment 63, wherein B2 is arginine, glycine, or valine.

### Embodiment 65

The pouch according to any one of embodiments 55-64, wherein P is p-methoxycarbonyl (Moc) or benzyloxycarbonyl (Cbz).

### Embodiment 66

The pouch according to embodiment 50, wherein the enzyme inhibitor is Cbz-Arg-Ala-Tyr-H, Ac-Gly-Ala-Tyr-H, Cbz-Gly-Ala-Tyr-H, Cbz-Gly-Ala-Tyr-CF3, Cbz-Gly-Ala-Leu-H, Cbz-Val-Ala-Leu-H, Cbz-Val-Ala-Leu-CF3, Moc-Val-Ala-Leu-CF3, Cbz-Gly-Ala-Phe-H, Cbz-Gly-Ala-Phe-CF3, Cbz-Gly-Ala-Val-H, Cbz-Gly-Gly-Tyr-H, Cbz-Gly-Gly-Phe-H, Cbz-Arg-Val-Tyr-H, Cbz-Leu-Val-Tyr-H, Ac-Leu-Gly-Ala-Tyr-H, Ac-Phe-Gly-Ala-Tyr-H, Ac-Tyr-Gly-Ala-Tyr-H, Ac-Phe-Gly-Ala-Leu-H, Ac-Phe-Gly-Ala-Phe-H, Ac-Phe-Gly-Val-Tyr-H, Ac-Phe-Gly-Ala-Met-H, Ac-Trp-Leu-Val-Tyr-H, MeO—CO-Val-Ala-Leu-H, MeNCO-Val-Ala-Leu-H, MeO—CO-Phe-Gly-Ala-Leu-H, MeO—CO-Phe-Gly-Ala-Phe-H, MeSO2-Phe-Gly-Ala-Leu-H, MeSO2-Val-Ala-Leu-H, PhCH2O—P(OH)(O)-Val-Ala-Leu-H, EtSO2-Phe-Gly-Ala-Leu-H, PhCH2SO2-Val-Ala-Leu-H, PhCH2O—P(OH)(O)-Leu-Ala-Leu-H, PhCH2O—P(OH)(O)-Phe-Ala-Leu-H, or MeO—P(OH)(O)-Leu-Gly-Ala-Leu-H or a hydrosulfite adduct of any of these, wherein Cbz is benzyloxycarbonyl and Moc is methoxycarbonyl.

### Embodiment 67

The pouch according to embodiment 50, wherein the enzyme inhibitor is Cbz-Arg-Ala-Tyr-H, Ac-Gly-Ala-Tyr-H, Cbz-Gly-Ala-Tyr-H, Cbz-Gly-Ala-Tyr-CF₃, Cbz-Gly-Ala-Leu-H, Cbz-Val-Ala-Leu-H, Cbz-Val-Ala-Leu-CF₃, Moc-Val-Ala-Leu-CF₃, Cbz-Gly-Ala-Phe-H, Cbz-Gly-Ala-Phe-CF₃, Cbz-Gly-Ala-Val-H, Cbz-Gly-Gly-Tyr-H, Cbz-Gly-Gly-Phe-H, Cbz-Arg-Val-Tyr-H, Cbz-Leu-Val-Tyr-H, MeO—CO-Val-Ala-Leu-H, MeNCO-Val-Ala-Leu-H, MeSO₂-Val-Ala-Leu-H, PhCH₂O—P(OH)(O)-Val-Ala-Leu-H, PhCH₂SO₂-Val-Ala-Leu-H, PhCH₂O—P(OH)(O)-Leu-Ala-Leu-H, or PhCH₂O—P(OH)(O)-Phe-Ala-Leu-H, or a hydrosulfite adduct of any of these, wherein Cbz is benzyloxycarbonyl and Moc is methoxycarbonyl.

### Embodiment 68

The pouch according to embodiment 50, wherein the enzyme inhibitor is Cbz-Gly-Ala-Tyr-H or Moc-Val-Ala-Leu-H, or a hydrosulfite adduct thereof, wherein Cbz is benzyloxycarbonyl and Moc is methoxycarbonyl.

### Embodiment 69

A method of preparing a pouch, the method comprising forming a wall which forms a chamber, the wall comprising a water-soluble polymer; providing a particulate solid comprising an enzyme, and a liquid comprising a surfactant and/or a polyol in the chamber; and sealing the chamber to form the pouch.

### Embodiment 70

The method according to embodiment 69, wherein the pouch is as defined in any one of embodiments 1-68.

### Embodiment 71

The method according to embodiment 69 or 70, which is performed using a form fill seal machine.

### Embodiment 72

A method of preparing an enzyme-containing detergent composition comprising adding a pouch to an aqueous composition, wherein the pouch comprises a wall forming a chamber, the wall comprising a water-soluble polymer, and the chamber containing a particulate solid comprising an enzyme and a liquid comprising a surfactant and/or a polyol.

### Embodiment 73

The method according to embodiment 72, wherein the pouch is added to an aqueous composition that comprises one or more detergent ingredients, such as surfactants and/or builders.

### Embodiment 74

The method according to embodiment 72 or 73, wherein the pouch is as defined in any one of embodiments 1-68.

### Embodiment 75

The method according to any one of embodiments 72-74, wherein the aqueous composition is agitated to aid the dissolution of the pouch.

### Embodiment 76

A detergent composition obtainable by the method of any one of embodiments 72-75.

### Embodiment 77

A detergent composition comprising a surfactant and/or a builder, PVA, and at least 50% water.

The present invention is further described by the following examples which should not be construed as limiting the scope of the invention.

### EXAMPLES

Preferably, an enzyme pouch is a PVA bag that contains a spray dried enzyme powder mixed with formulation ingredients.

It has been found that PVA film in particular is beneficial for the safety of the product.

One function of the optional formulation chemicals is to wet the spray dried product to avoid enzyme dust and facilitate the dosing and handling of enzyme powder. In addition, any such formulation chemicals should not dissolve the enzyme powder inside the PVA film. As PVA film is water soluble, a low water content (e.g., less than about 5%, preferably less than about 3%) in the enzyme pouch formulation is preferred.

Formulation Ingredients:

Spray dried powder of a protease enzyme concentrate

Softanol™ 90

PEG300 and PEG400

Protease inhibitor (also simply referred to as ‘inhibitor’ in the example): Cbz-Gly-Ala-NHCH(CH₂C₆H₄pOH)CH(OH)(SO₃)Na, wherein Cbz is benzyloxycarbonyl (hydrosulfite/bisulfite form of a peptide aldehyde).

The protease used in the Examples is Savinase™, which has the amino acid sequence as shown in SEQ ID NO: 1.

Pouch/Bag Production Procedure

A typical procedure for preparing an enzyme pouch/bag of the invention is shown in FIG. 1. Firstly, a liquid enzyme (e.g., protease, amylase, lipase, etc.) concentrate is spray-dried to give a powder. Then a wetted mixture is generated by uniformly mixing additives, stabilizers (such as polyol, surfactant, and/or inhibitor) and spray-dried powder together. The consistency of the wetted mixture may be quite sticky and viscous at this stage. Then, the wetted mixture is placed in a mold/upon a sheet of water-soluble film, such as a PVA film, and then the mixture of enzyme is sealed to form a pouch, for example with another sheet of film. Any desired amount of the wetted mixture may be contained in a pouch. Conveniently, from about 10-70% of the mixture can be on a solid form (as powder/particles). The water content of the mixture is preferably lower than 10% w/w, and if salt is added to provide a higher ionic strength, the water content can be up to 15%.

The following formulations were prepared and tested to evaluate the physical and enzymatic stability of enzyme-containing bags, Bag 1, Bag 2, Bag 3, and Bag 4.

TABLE 1
Formulation of mixture in enzyme bags (percentages are
w/w based on the formulation in the bags). The weight of
mixture in each bag is about 2-3 gram.

| | Bag 1 | Bag 2 | Bag 3 | Bag 4 |
|---|---|---|---|---|
| 50% w/w spray-dried protease | 95.4% | 47.7% | 47.7% | 100% |
| powder + 50% w/w Softanol ™ 90 | | | | |
| PEG300 | — | 50% | — | |
| PEG400 | — | — | 50% | |
| Inhibitor* | 1.01% | 0.50% | 0.50% | |
| MPG | 1.84% | 0.92% | 0.92% | |
| Water | <2% | <1% | <1% | |

### Example 1

Physical Stability of Mixture in Enzyme Bag Product after Storage

Different to the conventional liquid or solid products, an enzyme bag product of the invention has no haze or precipitation concern like a liquid product. There is also no enzyme dust issue like a conventional solid product. The physical stability of the enzyme bag is tightly related to the safety of the PVA bag and possible leakage of enzyme from the bag. After 13W storage at 40° C., enzyme bag 2 (shown in FIG. 2) looks similar to a fresh sample, but with a very slight color change. No leakage and no deformation of the PVA film were observed.

### Example 2

Enzymatic Stability of Mixture in Enzyme Bag Product after Storage

The enzyme mixture in bags (Bag 1, Bag 2, Bag 3) are highly stable, as shown in Table 2.

TABLE 2
Enzymatic stability of content mixture of Enzyme Bags

| | Residual protease activity | | |
|---|---|---|---|
| | after 13 weeks storage | | |
| | 25° C. | 40° C. | 50° C. |
| Bag 1 | 89% | 91% | 86% |
| Bag 2 | 99% | 98% | 85% |
| Bag 3 | 103% | 92% | 84% |

The residual activities were higher than 90% after storage at 40° C. for 13 weeks (13W40° C.), and about 80-90% after storage at 50° C. for 13 weeks (13W50° C.).

### Example 3

Dissolution of the Mixture in Enzyme Bag Product in Commercial Detergents

A dissolution test was conducted by dissolving the contents (‘mixtures’) of Bag 1-in 4ifferent detergents at room temperature with stirring by a magnetic rotor (600 rpm). Three detergents were chosen as representatives of liquid detergents in the emerging market (one is Chinese model detergent base (1% soap, 3.8% LAS, 0.4% TEA, 8% AES, 4% AEO9, 2% Sodium Citrate, 0.02% CaCl₂.H₂O, up to 100% with DI water pH 8), Detergent 1. Other two are Chinese commercial detergents, Detergent 2 and Detergent 3. Surfactants contained in all the three detergents meet the requirement of the Chinese national standard (total surfactants >15%).

The dosage of mixture (0.08% w/w) was equal to the higher dosage of relevant liquid enzyme product in the detergents (0.5% w/w).

TABLE 3
Time required to dissolve the content mixture of
Bag 4 in three detergents

| Dissolution time (h) | Detergent 1 | Detergent 2 | Detergent 3 |
|---|---|---|---|
| Stirring @RT | 2 | >24 | 12 |
| Stirring @40° C. | 1.45 | >24 | 3 |
| Stirring @60° C. | 0.5 | 5 | 1.5 |

* Dissolution time starts from the addition of the mixture until full dissolution without any haziness and precipitate.

The content mixture of Bag 4 required more than 2 hours to completely dissolve in the detergents when stirring at room temperature.

TABLE 4
Time required at room temperature to dissolve the
content mixture of Bag 1 to Bag 3 in three detergents

| | Mixture of Bag 1 | Mixture of Bag 2 or Bag 3 |
|---|---|---|
| Detergent 1 | 20 min | <5 min |
| Detergent 2 | 20 min | <5 min |
| Detergent 3 | 40 min | <5 min |

The mixture of Bag 1 required about 20-40 min to fully dissolve in the detergents. The mixtures of Bag 2 and Bag 3 (which contain PEG300 or PEG400 as shown in Table 1) dissolved considerably faster. The mixtures of Bag 2 and Bag 3 dissolved quickly in all the three detergents, within 5 min. Thus, the presence of particularly polyethylene glycol, but also an inhibitor, promoted a very fast dissolution of the content mixture of bags in the detergents.

### Example 4

Matrix Stability of Enzyme Bag in Commercial Detergents

Enzymatic stability of the contents (‘mixtures’) of Bag 1-3 was tested in three detergents. One was Chinese model detergent base, Detergent 1. Other two were Chinese commercial detergents, Detergent 2 and Detergent 3. Surfactants contained in all three detergents meet the requirement of the Chinese national standard (>15% surfactant). 1% Na-formate and 0.06% CaCl₂ were pre-added into the detergents as stabilizers.

Matrix stability means the enzymatic stability in detergents. The content mixtures of Bag 2 and Bag 3 were investigated in three detergents. The dosage of mixture (0.03% w/w) was equal to the normal dosage of relevant liquid enzyme product in the detergents (0.2% w/w).

TABLE 5
Matrix stability of Enzyme Bags in different detergents

| | Storage condition | Mixture of Bag 2 | Mixture of Bag 3 |
|---|---|---|---|
| Detergent 1 | 2 W 37° C. | 79% | 79% |
| | 4 W 37° C. | 69% | 68% |
| Detergent 2 | 2 W 37° C. | 98% | 98% |
| | 4 W 37° C. | 87% | 89% |
| Detergent 3 | 2 W 37° C. | 77% | 80% |
| | 4 W 37° C. | 66% | 63% |

After 4W 37° C. storage, the residual activities of enzyme in the detergent were high enough to be considered acceptable in the industry. The enzymatic stabilities of the mixtures in the liquid detergents were at least equal to conventional liquid enzyme product formulations.

1. A pouch comprising a wall forming a chamber, the wall comprising a water-soluble polymer, and the chamber containing a particulate solid comprising an enzyme or a microorganism, and a liquid comprising a surfactant and/or a polyol.

2. The pouch according to claim 1, wherein the wall comprises PVA.

3. The pouch according to claim 1, which weighs at least 50 g.

4. The pouch according to claim 1, wherein the content of the chamber contains at least 10% w/w of particulate solid.

5. The pouch according to claim 1, which comprises at least 1% w/w active enzyme protein.

6. The pouch according to claim 1, wherein the enzyme is selected from a protease, subtilisin, metalloprotease, lipase, cutinase, amylase, carbohydrase, cellulase, pectinase, mannanase, arabinase, galactanase, xanthanase, xylanase, DNase, perhydrolase, oxidoreductase, laccase, peroxidase, peroxygenase, and haloperoxidase.

7. The pouch according to claim 1, wherein the microorganism is a bacterial spore.

8. The pouch according to claim 1, wherein the polyol comprises glycerol, (mono, di, or tri) propylene glycol, sugar alcohol, polypropylene glycol, and/or polyethylene glycol, with a molecular weight in the range of 200-800.

9. The pouch according to claim 1, wherein the surfactant comprises a non-ionic surfactant.

10. The pouch according to claim 1, wherein the particulate solid comprises a spray dried enzyme.

11. The pouch according to claim 1, wherein the chamber further contains an enzyme inhibitor.

12. The pouch according to claim 11 wherein the enzyme is a protease and the inhibitor is either: a) a peptide aldehyde or a derivative thereof, or a boronic acid.

13. The pouch according to claim 11, wherein the protease inhibitor is Cbz-Arg-Ala-Tyr-H, Ac-Gly-Ala-Tyr-H, Cbz-Gly-Ala-Tyr-H, Cbz-Gly-Ala-Tyr-CF3, Cbz-Gly-Ala-Leu-H, Cbz-Val-Ala-Leu-H, Cbz-Val-Ala-Leu-CF3, Moc-Val-Ala-Leu-CF3, Cbz-Gly-Ala-Phe-H, Cbz-Gly-Ala-Phe-CF3, Cbz-Gly-Ala-Val-H, Cbz-Gly-Gly-Tyr-H, Cbz-Gly-Gly-Phe-H, Cbz-Arg-Val-Tyr-H, Cbz-Leu-Val-Tyr-H, Ac-Leu-Gly-Ala-Tyr-H, Ac-Phe-Gly-Ala-Tyr-H, Ac-Tyr-Gly-Ala-Tyr-H, Ac-Phe-Gly-Ala-Leu-H, Ac-Phe-Gly-Ala-Phe-H, Ac-Phe-Gly-Val-Tyr-H, Ac-Phe-Gly-Ala-Met-H, Ac-Trp-Leu-Val-Tyr-H, MeO—CO-Val-Ala-Leu-H, MeNCO-Val-Ala-Leu-H, MeO—CO-Phe-Gly-Ala-Leu-H, MeO—CO-Phe-Gly-Ala-Phe-H, MeSO2-Phe-Gly-Ala-Leu-H, MeSO2-Val-Ala-Leu-H, PhCH2O—P(OH)(O)-Val-Ala-Leu-H, EtSO2-Phe-Gly-Ala-Leu-H, PhCH2SO2-Val-Ala-Leu-H, PhCH2O—P(OH)(O)-Leu-Ala-Leu-H, PhCH2O—P(OH)(O)-Phe-Ala-Leu-H, or MeO—P(OH)(O)-Leu-Gly-Ala-Leu-H or a hydrosulfite adduct thereof, wherein Cbz is benzyloxycarbonyl and Moc is methoxycarbonyl.

14. A method of preparing a pouch according to claim 1, the method comprising forming a wall which forms a chamber, the wall comprising a water-soluble polymer; providing a particulate solid comprising an enzyme or a microorganism, and a liquid comprising a surfactant and/or a polyol in the chamber; and sealing the chamber to form the pouch.

15. A method of preparing an enzyme-containing detergent composition, comprising adding a pouch according to claim 1 to an aqueous composition comprising one or more detergent ingredients, such as surfactants and/or builders.

16. (canceled)

17. The detergent composition of claim 16, comprising a surfactant and/or a builder, PVA, and at least 50% water.

18. The pouch according to claim 11, wherein the protease inhibitor is Cbz-Gly-Ala-Tyr-H or Moc-Val-Ala-Leu-H, or a hydrosulfite adduct thereof, and wherein Cbz is benzyloxycarbonyl and Moc is methoxycarbonyl.

19. The pouch according to claim 1, wherein the pouch weighs from 50 g to 25 kg, wherein the microorganism is a Bacillus endospore, wherein the polyol comprises polyethylene glycol or polypropylene glycol with a molecular weight in the range of 200-800, and wherein the surfactant is a polyoxyethylene alkyl ether surfactant.

20. The pouch according to claim 6, wherein the pouch weighs from 50 g to 25 kg, wherein the microorganism is a Bacillus endospore, wherein the polyol comprises polyethylene glycol or polypropylene glycol with a molecular weight in the range of 200-800, and wherein the surfactant is a polyoxyethylene alkyl ether surfactant.

21. The pouch according to claim 13, wherein the pouch weighs from 50 g to 25 kg, wherein the microorganism is a Bacillus endospore, wherein the polyol comprises polyethylene glycol or polypropylene glycol with a molecular weight in the range of 200-800, and wherein the surfactant is a polyoxyethylene alkyl ether surfactant.


SEQUENCE LISTING (10 sequences)

SEQ ID NO: 1 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 2 - protein, 4 aa, Artificial
  Subtilisin inhibitor
  MISC_FEATURE (1)..(1): Acetyl-Leu
  MISC_FEATURE (4)..(4): Tyr-H
LGAY

SEQ ID NO: 3 - protein, 4 aa, Artificial
  Subtilisin inhibitor
  MISC_FEATURE (1)..(1): Acetyl-Phe
  MISC_FEATURE (4)..(4): Tyr-H
FGAY

SEQ ID NO: 4 - protein, 4 aa, Artificial
  Subtilisin inhibitor
  MISC_FEATURE (1)..(1): Acetyl-Tyr
  MISC_FEATURE (4)..(4): Tyr-H
YGAY

SEQ ID NO: 5 - protein, 4 aa, Artificial
  Subtilisin inhibitor
  MISC_FEATURE (1)..(1): Acetyl-Phe; MeO-CO-Phe; MeSO2-Phe; or EtSO2-Phe
  MISC_FEATURE (4)..(4): Leu-H
FGAL

SEQ ID NO: 6 - protein, 4 aa, Artificial
  Subtilisin inhibitor
  MISC_FEATURE (1)..(1): Acetyl-Phe or MeO-CO-Phe
  MISC_FEATURE (4)..(4): Tyr-H
FGAF

SEQ ID NO: 7 - protein, 4 aa, Artificial
  Subtilisin inhibitor
  MISC_FEATURE (1)..(1): Acetyl-Phe
  MISC_FEATURE (4)..(4): Tyr-H
FGVY

SEQ ID NO: 8 - protein, 4 aa, Artificial
  Subtilisin inhibitor
  MISC_FEATURE (1)..(1): Acetyl-Phe
  MISC_FEATURE (4)..(4): Met-H
FGAM

SEQ ID NO: 9 - protein, 4 aa, Artificial
  Subtilisin inhibitor
  MISC_FEATURE (1)..(1): Acetyl-Trp
  MISC_FEATURE (4)..(4): Tyr-H
WLVY

SEQ ID NO: 10 - protein, 4 aa, Artificial
  Subtilisin inhibitor
  MISC_FEATURE (1)..(1): MeO-P(OH)(O)-Leu
  MISC_FEATURE (4)..(4): Leu-H
LGAL